(19) United States
(12) Patent Application Publication
(10) Pub. No.: US 2026/0274907 A1
(43) Pub. Date: Sep. 17, 2026

(54) WOOLLY MAMMOTH SPECIFIC GENE VARIANTS AND COMPOSITIONS COMPRISING SAME

(71) Applicant: Colossal Biosciences Inc., Austin, TX (US)

(72) Inventors: Eriona HYSOLLI, Austin, TX (US); Austin BOW, Austin, TX (US); Michael ABRAMS, Austin, TX (US); Ben LAMM, Austin, TX (US); Devon FITZPATRICK, Austin, TX (US)

(21) Appl. No.: 19/473,083

(22) PCT Filed: Apr. 5, 2024
(86) PCT No.: PCT/US2024/023197

§ 371 Date: Oct. 6, 2025

Related U.S. Application Data
(60) Provisional application No. 63/494,762, filed on Apr. 6, 2023.

(51) Int. Cl. C07K 14/47; A01K 67/0275; C12N 9/10; C12N 9/78

(52) U.S. Cl. CPC C07K 14/47; A01K 67/0275; C12N 9/1029; C12N 9/78; C12Y 203/02013; C12Y 305/04004; A01K 2217/052; A01K 2217/054; A01K 2227/10

(57) ABSTRACT

Provided herein are isolated nucleic acids, vectors, recombinant cells, and transgenic animals capable of expressing at least one or more woolly mammoth genes. Also provided are recombinant cells and transgenic animals that comprise one or more woolly mammoth specific deletions of regulatory regions upstream of identified transcription start sites.

### CROSS REFERENCE TO RELATED APPLICATION

This application claims priority to U.S. Provisional Application No. 63/494,762, filed Apr. 6, 2023, the disclosure of which is herein incorporated by reference in its entirety.

### FIELD OF THE INVENTION

The present disclosure generally relates to gene edited, and/or reprogrammed mammalian cells and uses thereof.

### REFERENCE TO SEQUENCE LISTING SUBMITTING ELECTRONICALLY

This application contains a sequence listing, which is submitted electronically. The contents of the electronic sequence listing (069296.9WO1 Sequence Lisitng.xml; size: 1,857,418 bytes; and creation date of Mar. 29, 2024) is herein incorporated by reference in its entirety.

### BACKGROUND OF THE INVENTION

Currently, there is an unmet need for the development of elephant tissue cultures, genome editing of non-human cells, and biological tools to aid in animal conservation efforts as well as animal de-extinction efforts. Synthetic biology and gene editing can improve treatments for wildlife diseases and rectify ecological imbalances caused by climate change, pollution, human consumption, hunting, human-caused disturbances, depletion of resources, deforestation, and extinction events. Creating and biobanking of tissues and cells lines from endangered and extinct species can preserve them for future research and can help to repopulate the endangered and extinct species. However, currently, there is a lack of tissues and cells lines to be used for the de-extinction processes for animals that are extinct.

### BRIEF SUMMARY OF THE INVENTION

Provided herein are isolated nucleic acid sequences comprising a nucleotide sequence having at least 80%, at least 85%, at least 90%, at least 95%, at least 96%, at least 97%, at least 98%, or at least 99% identity to at least one of G protein-coupled receptor 98 (GPR98) (SEQ ID NO:1), microtubule actin crosslinking factor 1 (MACF1) (SEQ ID NO:2), adenosine deaminase RNA specific B2 (ADARB2) (SEQ ID NO:3), centrosomal protein 290 (CEP290) (SEQ ID NO:4), keratin 4 (KRT4) (SEQ ID NO:5), LOC126076011 (SEQ ID NO:6), NCK associated protein 5 (NCKAP5) (SEQ ID NO:7), laminin subunit beta 4 (LAMB4) (SEQ ID NO:8), Niemann-Pick C1-like 1 (NPC1L1) (SEQ ID NO:9), adhesion G protein-coupled receptor D2 (ADGRD2) (SEQ ID NO:10), ninjurin 1 (NINJ1) (SEQ ID NO:11), AH4NAK nucleoprotein 2 (AHNAK2) (SEQ ID NO: 12), cation channel sperm associated auxiliary subunit beta (CATSPERB) (SEQ ID NO:13), pecanex-like 4 (PCNXL4) (SEQ ID NO: 14), spectrin repeat containing nuclear envelope protein 2 (SYNE2) (SEQ ID NO:15), NLR family pyrin domain containing 12 (NLRP12) (SEQ ID NO: 16), LOC126086768 (SEQ ID NO:17), WD repeat domain 90 (WDR90) (SEQ ID NO:18), breast cancer 2 (BRCA2) (SEQ ID NO:19), protein kinase, DNA-activated catalytic subunit (PRKDC) (SEQ ID NO:20), vacuolar protein sorting 13 homolog B (VPS13B) (SEQ ID NO:21), prosaposin (PSAP) (SEQ ID NO:22), SEC31 homolog B (SEC31B) (SEQ ID NO:23), keratin 28 (KRT28) (SEQ ID NO:24), keratin 35 (KRT35) (SEQ ID NO:25), keratin 40 (KRT40) (SEQ ID NO:26), myosin heavy chain 4 (MYH4) (SEQ ID NO:27), phosphoinositide interacting regulator of TRP (PIRT) (SEQ ID NO:28), polycystin 1 like 2 (PKDIL2) (SEQ ID NO:29), retinitis pigmentosa 1-like protein (RP1L1) (SEQ ID NO:30), chromosome X open reading frame 58 (CXorf58) (SEQ ID NO:31), LOC126068772 (SEQ ID NO:32), LOC126069872 (SEQ ID NO:33), thyroid hormone receptor associated protein 3 (THRAP3) (SEQ ID NO:34), centromere protein C 1 (CENPC1) (SEQ ID NO:35), dentinogenesis and dentin sialophosphoprotein (DSPP) (SEQ ID NO:36), fibroblast growth factor 5 (FGF5) (SEQ ID NO:37), cation channel sperm associated auxiliary subunit gamma (CATSPERG) (SEQ ID NO:38), myosin heavy chain 1 (MYH1) (SEQ ID NO:39), myosin heavy chain 13 (MYH13) (SEQ ID NO:40), ATR interacting protein (ATRIP) (SEQ ID NO:41), and transglutaminase 3 (TGM3) (SEQ ID NO:42).

In certain embodiments, the nucleotide sequence comprises at least one of GPR98 (SEQ ID NO:1), MACF1 (SEQ ID NO:2), ADARB2 (SEQ ID NO:3), CEP290 (SEQ ID NO:4), KRT4 (SEQ ID NO:5), LOC126076011 (SEQ ID NO:6), NCKAP5 (SEQ ID NO:7), LAMB4 (SEQ ID NO:8), NPC1L1 (SEQ ID NO:9), ADGRD2 (SEQ ID NO:10), NINJ1 (SEQ ID NO:11), AHNAK2 (SEQ ID NO:12), CATSPERB (SEQ ID NO:13), PCNXL4 (SEQ ID NO:14), SYNE2 (SEQ ID NO:15), NLRP12 (SEQ ID NO:16), LOC126086768 (SEQ ID NO:17), WDR90 (SEQ ID NO:18), BRCA2 (SEQ ID NO:19), PRKDC (SEQ ID NO:20), VPS13B (SEQ ID NO:21), PSAP (SEQ ID NO:22), SEC31B (SEQ ID NO:23), KRT28 (SEQ ID NO:24), KRT35 (SEQ ID NO:25), KRT40 (SEQ ID NO:26), MYH4 (SEQ ID NO:27), PIRT (SEQ ID NO:28), PKD1L2 (SEQ ID NO:29), RP1L1 (SEQ ID NO:30), CXorf58 (SEQ ID NO:31), LOC126068772 (SEQ ID NO:32), LOC126069872 (SEQ ID NO:33), THRAP3 (SEQ ID NO:34), CENPC1 (SEQ ID NO:35), DSPP (SEQ ID NO:36), FGF5 (SEQ ID NO:37), CATSPERG (SEQ ID NO:38), MYH1 (SEQ ID NO:39), MYH13 (SEQ ID NO:40), ATRIP (SEQ ID NO:41), and TGM3 (SEQ ID NO:42).

Also provided are isolated vectors comprising the isolated nucleic acid sequences described herein.

Also provided are recombinant host cells comprising a nucleotide sequence having at least 80%, at least 85%, at least 90%, at least 95%, at least 96%, at least 97%, at least 98%, or at least 99% identity to at least one of GPR98 (SEQ ID NO:1), MACF1 (SEQ ID NO:2), ADARB2 (SEQ ID NO:3), CEP290 (SEQ ID NO:4), KRT4 (SEQ ID NO:5), LOC126076011 (SEQ ID NO:6), NCKAP5 (SEQ ID NO:7), LAMB4 (SEQ ID NO:8), NPC1L1 (SEQ ID NO:9), ADGRD2 (SEQ ID NO:10), NINJ1 (SEQ ID NO: 11), AHNAK2 (SEQ ID NO:12). CATSPERB (SEQ ID NO:13), PCNXL4 (SEQ ID NO: 14), SYNE2 (SEQ ID NO:15), NLRP12 (SEQ ID NO:16), LOC126086768 (SEQ ID NO:17), WDR90 (SEQ ID NO:18), BRCA2 (SEQ ID NO:19), PRKDC (SEQ ID NO:20), VPS13B (SEQ ID NO:21), PSAP (SEQ ID NO:22), SEC31B (SEQ ID NO:23), KRT28 (SEQ ID NO:24), KRT35 (SEQ ID NO:25), KRT40 (SEQ ID NO:26), MYH4 (SEQ ID NO:27), PIRT (SEQ ID NO:28), PKD1L2 (SEQ ID NO:29), RP1L1 (SEQ ID NO:30), CXorf58 (SEQ ID NO:31), LOC126068772 (SEQ ID NO:32), LOC126069872 (SEQ ID NO:33), THRAP3 (SEQ ID NO:34), CENPC1 (SEQ ID NO:35), DSPP (SEQ ID NO:36), FGF5 (SEQ ID NO:37), CATSPERG (SEQ ID NO:38), MYH1 (SEQ ID NO:39), MYH13 (SEQ ID NO:40), ATRIP (SEQ ID NO:41), and TGM3 (SEQ ID NO:42). In certain embodiments, the recombinant host cell comprises a nucleotide sequence having at least one of GPR98 (SEQ ID NO:1), MACF1 (SEQ ID NO:2), ADARB2 (SEQ ID NO:3), CEP290 (SEQ ID NO:4), KRT4 (SEQ ID NO:5), LOC126076011 (SEQ ID NO:6), NCKAP5 (SEQ ID NO:7), LAMB4 (SEQ ID NO:8), NPC1L1 (SEQ ID NO:9), ADGRD2 (SEQ ID NO:10), NINJ1 (SEQ ID NO:11), AHNAK2 (SEQ ID NO:12), CATSPERB (SEQ ID NO:13), PCNXL4 (SEQ ID NO: 14), SYNE2 (SEQ ID NO:15), NLRP12 (SEQ ID NO:16), LOC126086768 (SEQ ID NO: 17), WDR90 (SEQ ID NO:18), BRCA2 (SEQ ID NO:19), PRKDC (SEQ ID NO:20), VPS13B (SEQ ID NO:21), PSAP (SEQ ID NO:22), SEC31B (SEQ ID NO:23), KRT28 (SEQ ID NO:24), KRT35 (SEQ ID NO:25), KRT40 (SEQ ID NO:26), MYH4 (SEQ ID NO:27), PIRT (SEQ ID NO:28), PKD1L2 (SEQ ID NO:29), RP1L1 (SEQ ID NO:30), CXorf58 (SEQ ID NO:31), LOC126068772 (SEQ ID NO:32), LOC126069872 (SEQ ID NO:33), THRAP3 (SEQ ID NO:34), CENPC1 (SEQ ID NO:35), DSPP (SEQ ID NO:36), FGF5 (SEQ ID NO:37), CATSPERG (SEQ ID NO:38), MYH1 (SEQ ID NO:39), MYH13 (SEQ ID NO:40), ATRIP (SEQ ID NO:41), and TGM3 (SEQ ID NO:42).

Also provided are recombinant host cells comprising at least one of the isolated nucleic acid sequences described herein. In certain embodiments, the recombinant host cell comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 of the isolated nucleic acids described herein.

Also provided herein are recombinant host cells comprising a deletion of at least one nucleotide sequence upstream of a transcription start site of LOC126071805, WASH complex subunit 4 (WASHC4), LOC126075532, LOC126075533, alpha-fetoprotein (AFP), LOC126079103, LOC126079327, LOC126079327, LOC126080333, LOC126080367, LOC126080369, LOC126080369, LOC126080733, distal-less homeobox 6 (DLX6), extracellular matrix protein 2 (ECM2), LOC126085059, LOC126085481, LOC126086151, LOC126086152, LOC126086293, LOC126058227, LOC126058390, LOC126058396, forkhead box H1 (FOXH1), LOC126060018, LOC126060570, coiled-coil domain containing 15 (CCDC15), INO80 complex subunit B (INO80B), LOC126062579, LOC126063153, LOC126063990, LOC126063991, LOC126066513, LOC126066877, acyl-CoA wax alcohol acyltransferase 1 (AWAT1), transmembrane protein 187 (TMEM187), and LOC126069912, wherein the deleted nucleic acid sequence upstream of (a) LOC126071805 comprises SEQ ID NO:43, (b) WASHC4 comprises SEQ ID NO:44, (c) LOC126075532 comprises SEQ ID NO:45, (d) LOC126075533 comprises SEQ ID NO:46, (e) AFP comprises SEQ ID NO:47, (f) LOC126079103 comprises SEQ ID NO:48, (g) LOC126079327 comprises SEQ ID NO:49, (h) LOC126079327 comprises SEQ ID NO:50, (i) LOC126080333 comprises SEQ ID NO:51, (j) LOC126080367 comprises SEQ ID NO:52, (k) LOC126080369 comprises SEQ ID NO:53, (l) LOC126080369 comprises SEQ ID NO:54, (m) LOC126080733 comprises SEQ ID NO:55, (n) DLX6 comprises SEQ ID NO:56, (o) ECM2 comprises SEQ ID NO:57, (p) LOC126085059 comprises SEQ ID NO:58, (q) LOC126085481 comprises SEQ ID NO:59, (r) LOC126086151 comprises SEQ ID NO:60, (s) LOC126086152 comprises SEQ ID NO:61, (t) LOC126086293 comprises SEQ ID NO:62, (u) LOC126058227 comprises SEQ ID NO:63, (v) LOC126058390 comprises SEQ ID NO:64, (w) LOC126058396 comprises SEQ ID NO:65, (x) FOXH1 comprises SEQ ID NO:66, (y) LOC126060018 comprises SEQ ID NO:67, (z) LOC126060570 comprises SEQ ID NO:68, (aa) CCDC15 comprises SEQ ID NO:69, (bb) INO80B comprises SEQ ID NO:70, (cc) LOC126062579 comprises SEQ ID NO:71, (dd) LOC126063153 comprises SEQ ID NO:72, (ee) LOC126063990 comprises SEQ ID NO:73, (ff) LOC126063991 comprises SEQ ID NO:74, (gg) LOC126066513 comprises SEQ ID NO:75, (hh) LOC126066877 comprises SEQ ID NO:76, (ii) AWAT1 comprises SEQ ID NO:77, (jj) TMEM187 comprises SEQ ID NO:78, and (kk) LOC126069912 comprises SEQ ID NO:79.

In certain embodiments, the recombinant host cells comprising the isolated nucleic acids described herein further comprise a deletion of at least one nucleotide sequence upstream of a transcription start site of LOC126071805, WASHC4, LOC126075532, LOC126075533, AFP, LOC126079103, LOC126079327, LOC126079327, LOC126080333, LOC126080367, LOC126080369, LOC126080369, LOC126080733, DLX6, ECM2, LOC126085059, LOC126085481, LOC126086151, LOC126086152, LOC126086293, LOC126058227, LOC126058390, LOC126058396, FOXH1, LOC126060018, LOC126060570, CCDC15, INO80B, LOC126062579, LOC126063153, LOC126063990, LOC126063991, LOC126066513, LOC126066877, AWAT1, TMEM187, and LOC126069912, wherein the deleted nucleic acid sequence upstream of (a) LOC126071805 comprises SEQ ID NO:43, (b) WASHC4 comprises SEQ ID NO:44, (c) LOC126075532 comprises SEQ ID NO:45, (d) LOC126075533 comprises SEQ ID NO:46, (e) AFP comprises SEQ ID NO:47, (f) LOC126079103 comprises SEQ ID NO:48, (g) LOC126079327 comprises SEQ ID NO:49, (h) LOC126079327 comprises SEQ ID NO:50, (i) LOC126080333 comprises SEQ ID NO:51, (j) LOC126080367 comprises SEQ ID NO:52, (k) LOC126080369 comprises SEQ ID NO:53, (l) LOC126080369 comprises SEQ ID NO:54, (m) LOC126080733 comprises SEQ ID NO:55, (n) DLX6 comprises SEQ ID NO:56, (o) ECM2 comprises SEQ ID NO:57, (p) LOC126085059 comprises SEQ ID NO:58, (q) LOC126085481 comprises SEQ ID NO:59, (r) LOC126086151 comprises SEQ ID NO:60, (s) LOC126086152 comprises SEQ ID NO:61, (t) LOC126086293 comprises SEQ ID NO:62, (u) LOC126058227 comprises SEQ ID NO:63, (v) LOC126058390 comprises SEQ ID NO:64, (w) LOC126058396 comprises SEQ ID NO:65, (x) FOXH1 comprises SEQ ID NO:66, (y) LOC126060018 comprises SEQ ID NO:67, (z) LOC126060570 comprises SEQ ID NO: 68, (aa) CCDC15 comprises SEQ ID NO:69, (bb) INO80B comprises SEQ ID NO:70, (cc) LOC126062579 comprises SEQ ID NO:71, (dd) LOC126063153 comprises SEQ ID NO:72. (ee) LOC126063990 comprises SEQ ID NO:73, (ff) LOC126063991 comprises SEQ ID NO:74, (gg) LOC126066513 comprises SEQ ID NO:75, (hh) LOC126066877 comprises SEQ ID NO:76, (ii) AWAT1 comprises SEQ ID NO:77, (jj) TMEM187 comprises SEQ ID NO:78, and (kk) LOC126069912 comprises SEQ ID NO:79.

In certain embodiments, the recombinant host cell is a stem cell. The stem cell can, for example, be selected from an induced stem cell, embryonic stem (ES) cell, or a mesenchymal stem cell (MSC). In certain embodiments, the recombinant host cell is a reprogrammed cell. In certain embodiments, the recombinant host cell is a fibroblast cell or a mesenchymal cell. In certain embodiments, the recombinant host cell is selected from the group consisting of a nerve cell, a cartilage cell, a bone cell, a muscle cell, a bone cell, a fat cell, and an epidermal cell.

In certain embodiments, the recombinant host cell fails to express an endogenous homologue of at least one of GPR98, MACF1, ADARB2, CEP290, KRT4, LOC126076011, NCKAP5, LAMB4, NPC1L1, ADGRD2, NINJ1, AHNAK2, CATSPERB, PCNXL4, SYNE2, NLRP12, LOC126086768, WDR90, BRCA2, PRKDC, VPS13B, PSAP, SEC31B, KRT28, KRT35, KRT40, MYH4, PIRT, PKD1L2, RP1L1, CXorf58, LOC126068772, LOC126069872, THRAP3, CENPC1, DSPP, FGF5, CATSPERG, MYH1, MYH13, ATRIP, and TGM3. The recombinant host cell can, for example, fail to express the endogenous homologue of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 of GPR98, MACF1, ADARB2, CEP290, KRT4, LOC126076011, NCKAP5, LAMB4, NPC1L1, ADGRD2, NINJ1, AHNAK2, CATSPERB, PCNXL4, SYNE2, NLRP12, LOC126086768, WDR90, BRCA2, PRKDC, VPS13B, PSAP, SEC31B, KRT28, KRT35, KRT40, MYH4, PIRT, PKDIL2, RP1L1, CXorf58, LOC126068772, LOC126069872, THRAP3, CENPC1, DSPP, FGF5, CATSPERG, MYH1, MYH13, ATRIP, and TGM3.

In certain embodiments, the recombinant host cell is an elephant cell. The elephant cell can, for example, be selected from an Asian elephant cell (Elephas maximus), an African elephant cell (Loxodonta africana), an African forest elephant cell (Loxodonta cyclotis), and a Bornean elephant cell (Elephas maximus borneensis).

Also provided are transgenic animals comprising a recombinant host cell as described herein. In certain embodiments, the transgenic animal is an Asian elephant (Elephas maximus), an African elephant (Loxodonta africana), an African forest elephant (Loxodonta cyclotis), or a Bornean elephant (Elephas maximus borneensis).

Also provided are transgenic animals comprising at least one woolly mammoth (Mammuthus primigenius) gene variant. The at least one woolly mammoth (Mammuthus primigenius) gene variant can, for example, be selected from the group consisting of GPR98 (SEQ ID NO:1), MACF1 (SEQ ID NO:2), ADARB2 (SEQ ID NO:3), CEP290 (SEQ ID NO:4), KRT4 (SEQ ID NO:5), LOC126076011 (SEQ ID NO:6), NCKAP5 (SEQ ID NO:7), LAMB4 (SEQ ID NO:8), NPC1L1 (SEQ ID NO:9), ADGRD2 (SEQ ID NO:10), NINJ1 (SEQ ID NO:11), AHNAK2 (SEQ ID NO:12), CATSPERB (SEQ ID NO:13), PCNXL4 (SEQ ID NO:14), SYNE2 (SEQ ID NO:15), NLRP12 (SEQ ID NO:16), LOC126086768 (SEQ ID NO:17), WDR90 (SEQ ID NO:18), BRCA2 (SEQ ID NO:19), PRKDC (SEQ ID NO:20), VPS13B (SEQ ID NO:21), PSAP (SEQ ID NO:22), SEC31B (SEQ ID NO:23), KRT28 (SEQ ID NO:24), KRT35 (SEQ ID NO:25), KRT40 (SEQ ID NO:26), MYH4 (SEQ ID NO:27), PIRT (SEQ ID NO:28), PKDIL2 (SEQ ID NO:29), RP1L1 (SEQ ID NO:30), CXorf58 (SEQ ID NO:31), LOC126068772 (SEQ ID NO:32), LOC126069872 (SEQ ID NO:33), THRAP3 (SEQ ID NO:34), CENPC1 (SEQ ID NO:35), DSPP (SEQ ID NO:36), FGF5 (SEQ ID NO:37), CATSPERG (SEQ ID NO:38), MYH1 (SEQ ID NO:39), MYH13 (SEQ ID NO:40), ATRIP (SEQ ID NO:41), and TGM3 (SEQ ID NO:42).

In certain embodiments, the transgenic animal further comprises a deletion of at least one nucleotide sequence upstream of a transcription start site of LOC126071805, WASHC4, LOC126075532, LOC126075533, AFP, LOC126079103, LOC126079327, LOC126079327, LOC126080333, LOC126080367, LOC126080369, LOC126080369, LOC126080733, DLX6. ECM2, LOC126085059, LOC126085481, LOC126086151, LOC126086152, LOC126086293, LOC126058227, LOC126058390, LOC126058396, FOXH1, LOC126060018, LOC126060570, CCDC15, INO80B, LOC126062579, LOC126063153, LOC126063990, LOC126063991, LOC126066513, LOC126066877, AWAT1, TMEM187, and LOC126069912, wherein the deleted nucleic acid sequence upstream of (a) LOC126071805 comprises SEQ ID NO:43, (b) WASHC4 comprises SEQ ID NO:44, (c) LOC126075532 comprises SEQ ID NO:45, (d) LOC126075533 comprises SEQ ID NO:46, (e) AFP comprises SEQ ID NO:47, (f) LOC126079103 comprises SEQ ID NO:48, (g) LOC126079327 comprises SEQ ID NO:49, (h) LOC126079327 comprises SEQ ID NO:50, (i) LOC126080333 comprises SEQ ID NO:51, (j) LOC126080367 comprises SEQ ID NO:52, (k) LOC126080369 comprises SEQ ID NO:53, (l) LOC126080369 comprises SEQ ID NO:54, (m) LOC126080733 comprises SEQ ID NO:55, (n) DLX6 comprises SEQ ID NO:56, (o) ECM2 comprises SEQ ID NO:57, (p) LOC126085059 comprises SEQ ID NO:58, (q) LOC126085481 comprises SEQ ID NO:59, (r) LOC126086151 comprises SEQ ID NO:60, (s) LOC126086152 comprises SEQ ID NO:61, (t) LOC126086293 comprises SEQ ID NO:62, (u) LOC126058227 comprises SEQ ID NO:63, (v) LOC126058390 comprises SEQ ID NO:64, (w) LOC126058396 comprises SEQ ID NO:65, (x) FOXH1 comprises SEQ ID NO:66, (y) LOC126060018 comprises SEQ ID NO:67, (z) LOC126060570 comprises SEQ ID NO:68, (aa) CCDC15 comprises SEQ ID NO:69, (bb) INO80B comprises SEQ ID NO:70, (cc) LOC126062579 comprises SEQ ID NO:71, (dd) LOC126063153 comprises SEQ ID NO:72. (ee) LOC126063990 comprises SEQ ID NO:73, (ff) LOC126063991 comprises SEQ ID NO:74, (gg) LOC126066513 comprises SEQ ID NO:75, (hh) LOC126066877 comprises SEQ ID NO:76, (ii) AWAT1 comprises SEQ ID NO:77, (jj) TMEM187 comprises SEQ ID NO:78, and (kk) LOC126069912 comprises SEQ ID NO:79.

In certain embodiments, the transgenic animal is an Asian elephant (Elephas maximus), an African elephant (Loxodonta africana), an African forest elephant (Loxodonta cyclotis), or a Bornean elephant (Elephas maximus borneensis).

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIGS. 1A-1D show the results of gene editing experiments for NCKAP5 and NINJ1 replacement. FIG. 1A is a graph demonstrating the first enrichment for NCKAP5 and NINJ1 replacement. FIG. 1B is a chart for the first enrichment for NCKAP5 and NINJ1 replacement. FIG. 1C is a graph demonstrating the second enrichment for NCKAP5 and NINJ1 replacement. FIG. 1D is a chart for the second enrichment for NCKAP5 and NINJ1 replacement. ICE score is the knockout score for the target, and KI is the knock-in efficiency of the correct mammoth variant.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

For clarity of disclosure, and not by way of limitation, the detailed description of the invention is divided into subsections that describe or illustrate certain features, embodiments, or applications of the present invention.

### Definitions

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

The words “right,” “left,” “lower,” and “upper” designate directions in the drawings to which reference is made.

It should also be understood that the terms “about,” “approximately,” “generally,” “substantially” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The terms “identical” or percent “identity,” in the context of two or more nucleic acids or polypeptide sequences refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch. J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al, (1990) J. Mol. Biol. 215: 403-410 and Altschul et al, (1997) Nucleic Acids Res. 25: 3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0), For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul. Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

As used herein, the term “vector” is a replicon in which another nucleic acid segment can be operably inserted so as to bring about the replication or expression of the segment.

As used herein, the term “host cell” refers to a cell comprising a nucleic acid molecule of the present disclosure, such as, for example an isolated vector comprising an isolated nucleic acid of the invention. The “host cell” can be any type of cell, e.g., a primary cell, a cell in culture, or a cell from a cell line. In one embodiment, a “host cell” is a cell transfected with a nucleic acid molecule of the invention. In another embodiment, a “host cell” is a progeny or potential progeny of such a transfected cell. A progeny of a cell may or may not be identical to the parent cell, e.g., due to mutations or environmental influences that can occur in succeeding generations or integration of the nucleic acid molecule into the host cell genome. A host cell can be, for example, any type of prokaryotic, eukaryotic, or archaeal cell. In some instances, the host cell is a bacterial cell. In some instances, the host cell is a mammalian cell.

The term “expression” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications.

As used herein, the terms “peptide.” “polypeptide.” or “protein” can refer to a molecule comprised of amino acids and can be recognized as a protein by those of skill in the art. The conventional one-letter or three-letter code for amino acid residues is used herein. The terms “peptide,” “polypeptide,” and “protein” can be used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The peptide sequences described herein are written according to the usual convention whereby the N-terminal region of the peptide is on the left and the C-terminal region is on the right. Although isomeric forms of the amino acids are known, it is the L-form of the amino acid that is represented unless otherwise expressly indicated.

The term “heterologous nucleic acid” or “heterologous polypeptide” refers to a nucleic acid or a polypeptide whose sequence is not identical to that of another nucleic acid or poly peptide naturally found in the same host cell or the same host. As use herein, the “heterologous nucleic acid” or “heterologous polypeptide” can be heterologous to the bacterial cell and/or the mammalian host.

As used herein, the term “transform” or “transformation” refers to the transfer of a nucleic acid fragment into a host cell, such as a host bacterial cell, resulting in genetically-stable inheritance. Host cells comprising the transformed nucleic acid fragment are referred to as “recombinant” or “transgenic” or “transformed” organisms.

As used herein, the term “isolated” means a biological component (such as a nucleic acid, peptide or protein) has been substantially separated, produced apart from, or purified away from other biological components of the organism in which the component naturally occurs, i.e., other chromosomal and extrachromosomal DNA and RNA, and proteins. Nucleic acids, peptides and proteins that have been “isolated” thus include nucleic acids and proteins purified by standard purification methods. “Isolated” nucleic acids, peptides and proteins can be part of a composition and still be isolated if the composition is not part of the native environment of the nucleic acid, peptide, or protein. The term also embraces nucleic acids, peptides and proteins prepared by recombinant expression in a host cell as well as chemically synthesized nucleic acids.

As used herein, “gene” refers to a nucleic acid comprising an open reading frame encoding a polypeptide, including both exon and (optionally) intron sequences.

As used herein, a “promoter” is an example of a transcriptional regulatory sequence and is specifically a nucleic acid sequence generally described as the proximal region of a gene located 5′ to the start codon. The transcription of an adjacent nucleic acid segment is initiated at the promoter region. A repressible promoter's rate of transcription decreases in response to a repressing agent. An inducible promoter's rate of transcription increases in response to an inducing agent. A constitutive promoter's rate of transcription is not specifically regulated, though it can vary under the influence of general metabolic conditions.

The term “gene product,” as used herein, refers to any product encoded by a nucleic acid sequence. Accordingly, a gene product may, for example, be a primary transcript, a mature transcript, a processed transcript, or a protein or peptide encoded by a transcript. Examples for gene products, accordingly, include mRNAs, rRNAs, hairpin RNAs (e.g., microRNAs, shRNAs, siRNAs, tRNAs), and peptides and proteins, for example, reporter proteins or therapeutic proteins.

As used herein, the term “stem cell” refers to a cell that can self-renew and differentiate to at least one more-differentiated or less developmentally-capable phenotype. The term “stem cell” encompasses stem cell lines, induced stem cells, non-human embryonic stem cells, pluripotent stem cells, multiopotent stem cells, amniotic stem cells, placental stem cells, or adult stem cells. An “induced stem cell” is one derived from a non-pluripotent cell induced to a less-differentiated or more developmentally-capable phenotype by introduction of one or more reprogramming factors or genes. As the term is used herein, an induced stem cell need not be pluripotent, but has the capacity to differentiate, under appropriate conditions, to more than one more-highly-differentiated phenotype. It should be understood that the capacity was not present prior to the introduction of reprogramming factors. An induced stem cell will express at least one stem cell marker not expressed by the parent cell prior to introduction of reprogramming factors. In this context, a stem cell marker is exclusive of a factor introduced by reprogramming. An induced pluripotent stem cell, or iPS cell, has the induced capacity to differentiate, under appropriate conditions, to a cell phenotype derived from each of the endoderm, mesoderm, and ectoderm germ layers.

The term “marker” as used herein is used to describe a characteristic and/or phenotype of a cell. Markers can be used for selection of cells comprising characteristics of interest and can vary with specific cells. Markers are characteristics, whether morphological, structural, functional or biochemical (enzymatic) characteristics of the cell of a particular cell type, or molecules expressed by the cell type. In one aspect, such markers are proteins. Such proteins can possess an epitope for antibodies or other binding molecules available in the art. However, a marker can consist of any molecule found in or on a cell, including, but not limited to, proteins (peptides and polypeptides), lipids, polysaccharides, nucleic acids and steroids. Examples of morphological characteristics or traits include, but are not limited to, shape, size, and nuclear to cytoplasmic ratio. Examples of functional characteristics or traits include, but are not limited to, the ability to adhere to particular substrates, ability to incorporate or exclude particular dyes, ability to migrate under particular conditions, and the ability to differentiate along particular lineages. Markers can be detected by any method available to one of skill in the art. Markers can also be the absence of a morphological characteristic or absence of proteins, lipids etc. Markers can be a combination of a panel of unique characteristics of the presence and/or absence of polypeptides and other morphological or structural characteristics. In one embodiment, the marker is a cell surface marker.

The term “exogenous” refers to a substance present in a cell that was introduced by the hand of man. The term “exogenous” when used herein can refer to a nucleic acid (e.g., a nucleic acid encoding a polypeptide) or a polypeptide that has been introduced by a process involving the hand of man into a biological system such as a cell or organism in which it is not normally found. Alternatively. “exogenous” can refer to a nucleic acid or a polypeptide that has been introduced by a process involving the hand of man into a biological system such as a cell or organism in which it is found in relatively lower amounts and in which one wishes to increase the amount of the nucleic acid or polypeptide in the cell or organism, e.g., to create ectopic expression or levels.

As used herein, the term “reprogramming genes” or “reprogramming factors” refers to agents or nucleic acid molecules that can induce the reprogramming process in a somatic cell to re-express a less-differentiated, more stem-cell like phenotype. The reprogramming factor can be a nucleic acid, a polypeptide, or a small molecule that promotes a reprogrammed phenotype when introduced to a cell. Non-limiting examples of reprogramming factors include: Oct4 (Octamer binding transcription factor-4), SOX2 (Sex determining region Y)-box 2, Klf4 (Kruppel Like Factor-4), and c-Myc. These are the so-called “classical” or “standard” set of reprogramming factors used to derive, for example, induced pluripotent stem cells. Additional factors that can be considered reprogramming factors when introduced in the process of reprogramming cells to a less differentiated or stem cell phenotype include LIN28+Nanog, Esrrb, Pax5 shRNA, C/EBPa, p53 siRNA, UTF1, DNMT shRNA, Wnt3a, SV40 LT(T), hTERT, small molecule chemical agents including, but not limited to BIX-01294, BayK8644, RG108, AZA, dexamethasone, VPA, TSA, SAHA, PD0325901+CHIR99021(2i) and A-83-01. In some embodiments, the reprogramming genes or factors are Oct4, Klf4, SOX2, and c-Myc.

As used herein, the terms “dedifferentiation” or “retrodifferentiation” or “reprogramming” refer to a process that generates a cell that re-expresses a less differentiated phenotype than the cell from which it is derived and/or expresses at least one stem cell marker not expressed prior to that process. For example, a terminally-differentiated cell can be dedifferentiated to a multipotent cell. That is, dedifferentiation shifts a cell backward along the differentiation spectrum of totipotent cells to fully differentiated cells. Typically, reversal of the differentiation phenotype of a cell requires artificial manipulation of the cell, for example, by introducing or expressing exogenous polypeptide factors. Reprogramming is not typically observed under native conditions in vivo or in vitro.

As used herein, a “reprogrammed cell” is a cell that has been contacted with one or more reprogramming factors and expresses a less differentiated phenotype than the cell from which it was derived. The reprogrammed cell can also have the capacity to self-renew and will express at least one stem cell marker that was not delivered to the cell as a reprogramming factor. Furthermore, the reprogrammed cell will have the capacity to differentiate into a more-differentiated somatic cell type following differentiation protocols provided herein or described in the art.

As used herein, the term “somatic cell” refers to any cell other than a germ cell, a cell present in or obtained from a pre-implantation embryo, or a cell resulting from proliferation of such a cell in vitro. Stated another way, a somatic cell refers to any cells forming the body of an organism, excluding germ cells. Every cell type in the mammalian body-apart from the sperm and ova and the cells from which they are made (gametocytes)—is a somatic cell: internal organs, skin, bones, blood, and connective tissue are all substantially made up of somatic cells. In some embodiments the somatic cell is a “non-embryonic somatic cell.” by which is meant a somatic cell that is not present in or obtained from an embryo and does not result from proliferation of such a cell in vitro. In some embodiments the somatic cell is an “adult somatic cell,” by which is meant a cell that is present in or obtained from an organism other than an embryo or a fetus or results from proliferation of such a cell in vitro.

### Nucleic Acids, Vectors, Recombinant Cells, and Transgenic Animals Expressing Woolly Mammoth Specific Variants

Woolly mammoths (Mammuthus primigenius) were cold-tolerant members of the elephant family that once ranged across the vast mammoth steppe of the Northern Hemisphere in the last ice age and became extinct across the majority of their range approximately 10,000 years ago. The woolly mammoth is arguably the best-characterized prehistoric animal, both through prehistoric art and from frozen remains found in Siberia and Alaska. These well-preserved specimens provide the rare opportunity to functionally characterize adaptive evolution in an extinct animal. Inhabitation of extreme environments, such as the cold regions of the northern latitudes, necessitates a suite of adaptive evolutionary changes. Genetic and morphological analyses of woolly mammoth specimens have revealed multiple physiological adaptations to cold, including dense, long hair, increased adipose tissue, decreased ears and tails, and hemoglobin structural polymorphisms. Studies of other cold-tolerant mammals have identified a number of convergent adaptations across the same genes and pathways, as well as unique adaptations to a shared environmental stressor.

The isolated nucleic acids, vectors, recombinant cells, and transgenic animals described herein are based, in part, on the discovery that cells (e.g., Elephas maximus and Loxodonta africana cells) can be modified to comprise and express alleles or homologues from the woolly mammoth (e.g., Mammuthus primigenius). In particular, viable cells can be gene-edited, whether by transfection, transduction or modification of existing elephant homologues to mimic the mammoth variants or alleles of the elephant genes. In some embodiments, the endogenous homologues of the mammoth genes are deleted or inactivated. Similar modifications to introduce woolly mammoth genes can be made to viable cells of other, non-human relatives of the elephant. The mammoth variants or alleles can modify the phenotype of the gene edited cells. The isolated nucleic acids, vectors, recombinant cells, and transgenic animals described herein provide a synthetic alternative to wildlife products and new tools for understanding genetic diversity and cellular biology in endangered and extinct species of wildlife.

In one aspect, described herein is at least one exogenous nucleic acid sequence encoding a woolly mammoth gene, or comprising a modification of an endogenous gene to express a woolly mammoth homologue or variant of the endogenous gene. Of particular interest are genes that are shared by every woolly mammoth genome sequenced, which are not shared by any elephant genome (Asian or African) sequenced. By choosing genes in this manner, effects of individual variation within the group of woolly mammoth genomes sequenced and variations in Asian and/or African elephant genomes are minimized to focus on those variant sequences that are fully mammoth. In view of this, as used herein, a “woolly mammoth gene,” “woolly mammoth gene variant” or “woolly mammoth homologue” is a gene encoding a polypeptide that has a sequence encoded by all woolly mammoth genomes sequenced, and which differs from the homologous polypeptide encoded in all African and Asian elephant genomes sequenced. In this context, “differs from” refers to a difference of at least one amino acid relative to the homologous polypeptides encoded by the African or Asian elephant. A non-coding or regulatory nucleic acid sequence can be considered a “woolly mammoth sequence” if a non-coding motif of at least 20 nucleotides is present in every woolly mammoth genome sequenced, and not present in any Asian or African elephant genome sequenced. An Asian or African elephant gene or sequence modified by human intervention to encode a woolly mammoth gene or gene variant sequence is a woolly mammoth gene or gene or gene variant as the term is used herein. Where a woolly mammoth gene or gene variant as referred to herein is only found encoded in a woolly mammoth genome, and where the woolly mammoth is extinct, a woolly mammoth gene or gene variant sequence is necessarily exogenous to a viable cell; that is, the woolly mammoth gene or gene variant sequence is “exogenous” whether the sequence is in the cell through introduction of a foreign sequence or through gene editing an endogenous sequence to encode the woolly mammoth gene or gene variant sequence.

Thus, provided herein are isolated nucleic acid sequences comprising woolly mammoth (Mammuthus primigenius) gene variants. The isolated nucleic acid sequences can, for example, comprise a nucleotide sequence having at least 80%, at least 85%, at least 90%, at least 95%, at least 96%, at least 97%, at least 98%, or at least 99% identity to at least one of G protein-coupled receptor 98 (GPR98) (SEQ ID NO: 1), microtubule actin crosslinking factor 1 (MACF1) (SEQ ID NO:2), adenosine deaminase RNA specific B2 (ADARB2) (SEQ ID NO:3), centrosomal protein 290 (CEP290) (SEQ ID NO:4), keratin 4 (KRT4) (SEQ ID NO:5), LOC126076011 (SEQ ID NO:6), NCK associated protein 5 (NCKAP5) (SEQ ID NO:7), laminin subunit beta 4 (LAMB4) (SEQ ID NO:8), Niemann-Pick C1-like 1 (NPC1L1) (SEQ ID NO:9), adhesion G protein-coupled receptor D2 (ADGRD2) (SEQ ID NO:10), ninjurin 1 (NINJ1) (SEQ ID NO:11), AHNAK nucleoprotein 2 (AHNAK2) (SEQ ID NO:12), cation channel sperm associated auxiliary subunit beta (CATSPERB) (SEQ ID NO:13), pecanex-like 4 (PCNXL4) (SEQ ID NO:14), spectrin repeat containing nuclear envelope protein 2 (SYNE2) (SEQ ID NO: 15), NLR family pyrin domain containing 12 (NLRP12) (SEQ ID NO:16), LOC126086768 (SEQ ID NO:17), WD repeat domain 90 (WDR90) (SEQ ID NO:18), breast cancer 2 (BRCA2) (SEQ ID NO:19), protein kinase, DNA-activated catalytic subunit (PRKDC) (SEQ ID NO:20), vacuolar protein sorting 13 homolog B (VPS13B) (SEQ ID NO:21), prosaposin (PSAP) (SEQ ID NO:22), SEC31 homolog B (SEC31B) (SEQ ID NO:23), keratin 28 (KRT28) (SEQ ID NO:24), keratin 35 (KRT35) (SEQ ID NO:25), keratin 40 (KRT40) (SEQ ID NO:26), myosin heavy chain 4 (MYH4) (SEQ ID NO:27), phosphoinositide interacting regulator of TRP (PIRT) (SEQ ID NO:28), polycystin 1 like 2 (PKD1L2) (SEQ ID NO:29), retinitis pigmentosa 1-like protein (RP1L1) (SEQ ID NO:30), chromosome X open reading frame 58 (CXorf58) (SEQ ID NO:31), LOC126068772 (SEQ ID NO:32), LOC126069872 (SEQ ID NO:33), thyroid hormone receptor associated protein 3 (THRAP3) (SEQ ID NO:34), centromere protein C 1 (CENPC1) (SEQ ID NO:35), dentinogenesis and dentin sialophosphoprotein (DSPP) (SEQ ID NO:36), fibroblast growth factor 5 (FGF5) (SEQ ID NO:37), cation channel sperm associated auxiliary subunit gamma (CATSPERG) (SEQ ID NO:38), myosin heavy chain 1 (MYH1) (SEQ ID NO:39), myosin heavy chain 13 (MYH13) (SEQ ID NO:40), ATR interacting protein (ATRIP) (SEQ ID NO:41), and transglutaminase 3 (TGM3) (SEQ ID NO:42).

In certain embodiments, the nucleotide sequence comprises at least one of GPR98 (SEQ ID NO:1), MACF1 (SEQ ID NO:2), ADARB2 (SEQ ID NO:3), CEP290 (SEQ ID NO:4), KRT4 (SEQ ID NO:5), LOC126076011 (SEQ ID NO:6), NCKAP5 (SEQ ID NO:7), LAMB4 (SEQ ID NO:8), NPC1L1 (SEQ ID NO:9), ADGRD2 (SEQ ID NO:10), NINJ1 (SEQ ID NO: 11), AHNAK2 (SEQ ID NO: 12), CATSPERB (SEQ ID NO: 13), PCNXL4 (SEQ ID NO:14), SYNE2 (SEQ ID NO:15), NLRP12 (SEQ ID NO:16), LOC126086768 (SEQ ID NO:17), WDR90 (SEQ ID NO:18), BRCA2 (SEQ ID NO:19), PRKDC (SEQ ID NO:20), VPS13B (SEQ ID NO:21), PSAP (SEQ ID NO:22), SEC31B (SEQ ID NO:23), KRT28 (SEQ ID NO:24), KRT35 (SEQ ID NO:25), KRT40 (SEQ ID NO:26), MYH4 (SEQ ID NO:27), PIRT (SEQ ID NO:28), PKD1L2 (SEQ ID NO:29), RP1L1 (SEQ ID NO:30), CXorf58 (SEQ ID NO:31), LOC126068772 (SEQ ID NO:32), LOC126069872 (SEQ ID NO:33), THRAP3 (SEQ ID NO:34), CENPC1 (SEQ ID NO:35), DSPP (SEQ ID NO:36), FGF5 (SEQ ID NO:37), CATSPERG (SEQ ID NO:38), MYH1 (SEQ ID NO:39), MYH13 (SEQ ID NO:40), ATRIP (SEQ ID NO:41), and TGM3 (SEQ ID NO:42).

In certain embodiments, the mammoth (Mammuthus primigenius) gene variant comprises a nucleotide sequence having at least 80%, at least 85%, at least 90%, or at least 95% identity to a nucleotide sequence selected from the group consisting of SEQ ID NOs: 1-42, and combinations thereof. The mammoth gene variant can, for example, comprise a nucleotide sequence having 80%, 81%, 82%, 83%, 84%, 85%, 86%, 87%, 88%, 89%, 90%, 91%, 92%, 93%, 94%, 95%, 96%, 97%, 98%, or 99% identity to a nucleotide sequence selected from the group consisting of SEQ ID NOs:1-42, and a combination thereof. The mammoth gene variant can, for example, comprise a nucleotide sequence selected from the group consisting of SEQ ID NOs:1-42, and combinations thereof.

In certain embodiments, the mammoth (Mammuthus primigenius) gene variant comprises at least one change in the nucleotide sequence of the gene. The change in the nucleotide sequence can, for example, be a substitution, an insertion, a deletion, or a combination thereof. The substitution, the insertion, the deletion, or a combination thereof can, for example, be in a 5′ untranslated region of the gene, an intron of the gene, an exon of the gene, a 3′ untranslated region of the gene, or a combination thereof. The substitution, the insertion, the deletion, or a combination thereof can, for example, be in a regulatory region of the gene.

Also provided are isolated vectors comprising the isolated nucleic acid sequences described herein.

Also provided are recombinant host cells or transgenic animals comprising a nucleotide sequence having at least 80%, at least 85%, at least 90%, at least 95%, at least 96%, at least 97%, at least 98%, or at least 99% identity to at least one of GPR98 (SEQ ID NO:1), MACF1 (SEQ ID NO:2), ADARB2 (SEQ ID NO:3), CEP290 (SEQ ID NO:4), KRT4 (SEQ ID NO:5), LOC126076011 (SEQ ID NO:6), NCKAP5 (SEQ ID NO:7), LAMB4 (SEQ ID NO:8), NPC1L1 (SEQ ID NO:9), ADGRD2 (SEQ ID NO:10), NINJ1 (SEQ ID NO:11), AHNAK2 (SEQ ID NO:12), CATSPERB (SEQ ID NO:13), PCNXL4 (SEQ ID NO:14), SYNE2 (SEQ ID NO:15), NLRP12 (SEQ ID NO:16), LOC126086768 (SEQ ID NO: 17), WDR90 (SEQ ID NO:18), BRCA2 (SEQ ID NO:19), PRKDC (SEQ ID NO:20), VPS13B (SEQ ID NO:21), PSAP (SEQ ID NO:22), SEC31B (SEQ ID NO:23), KRT28 (SEQ ID NO:24), KRT35 (SEQ ID NO:25), KRT40 (SEQ ID NO:26), MYH4 (SEQ ID NO:27), PIRT (SEQ ID NO:28), PKD1L2 (SEQ ID NO:29), RP1L1 (SEQ ID NO:30), CXorf58 (SEQ ID NO:31), LOC126068772 (SEQ ID NO:32), LOC126069872 (SEQ ID NO:33), THRAP3 (SEQ ID NO:34), CENPC1 (SEQ ID NO:35), DSPP (SEQ ID NO:36), FGF5 (SEQ ID NO:37), CATSPERG (SEQ ID NO:38), MYH1 (SEQ ID NO:39), MYH13 (SEQ ID NO:40), ATRIP (SEQ ID NO:41), and TGM3 (SEQ ID NO:42). In certain embodiments, the recombinant host cell or transgenic animal comprises a nucleotide sequence having at least one of GPR98 (SEQ ID NO:1), MACF1 (SEQ ID NO:2), ADARB2 (SEQ ID NO:3), CEP290 (SEQ ID NO:4), KRT4 (SEQ ID NO:5), LOC126076011 (SEQ ID NO:6), NCKAP5 (SEQ ID NO:7), LAMB4 (SEQ ID NO:8), NPC1L1 (SEQ ID NO:9), ADGRD2 (SEQ ID NO:10), NINJ1 (SEQ ID NO:11), AHNAK2 (SEQ ID NO: 12), CATSPERB (SEQ ID NO:13), PCNXL4 (SEQ ID NO:14), SYNE2 (SEQ ID NO:15), NLRP12 (SEQ ID NO:16), LOC126086768 (SEQ ID NO: 17), WDR90 (SEQ ID NO: 18), BRCA2 (SEQ ID NO: 19). PRKDC (SEQ ID NO:20), VPS13B (SEQ ID NO:21), PSAP (SEQ ID NO:22), SEC31B (SEQ ID NO:23), KRT28 (SEQ ID NO:24), KRT35 (SEQ ID NO:25), KRT40 (SEQ ID NO:26), MYH4 (SEQ ID NO:27), PIRT (SEQ ID NO:28), PKD1L2 (SEQ ID NO:29), RP1L1 (SEQ ID NO:30), CXorf58 (SEQ ID NO:31), LOC126068772 (SEQ ID NO:32). LOC126069872 (SEQ ID NO:33), THRAP3 (SEQ ID NO:34), CENPC1 (SEQ ID NO:35), DSPP (SEQ ID NO:36), FGF5 (SEQ ID NO:37), CATSPERG (SEQ ID NO:38), MYH1 (SEQ ID NO:39), MYH13 (SEQ ID NO:40), ATRIP (SEQ ID NO:41), and TGM3 (SEQ ID NO:42). In certain embodiments, the recombinant host cell comprises 2, 3, 4, 5, 6, 7, 8, 9, 10. 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 of the isolated nucleic acids described herein.

Also provided are recombinant host cells or transgenic animals comprising at least one of the isolated nucleic acid sequences described herein. In certain embodiments, the recombinant host cell or transgenic animal comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 of the isolated nucleic acids described herein.

Also provided herein are recombinant host cells or transgenic animals comprising a deletion of at least one nucleotide sequence upstream of a transcription start site of LOC126071805, WASH complex subunit 4 (WASHC4), LOC126075532, LOC126075533, alpha-fetoprotein (AFP), LOC126079103, LOC126079327, LOC126079327, LOC126080333, LOC126080367, LOC126080369, LOC126080369, LOC126080733, distal-less homeobox 6 (DLX6), extracellular matrix protein 2 (ECM2), LOC126085059, LOC126085481, LOC126086151, LOC126086152, LOC126086293, LOC126058227, LOC126058390, LOC126058396, forkhead box H1 (FOXH1), LOC126060018, LOC126060570, coiled-coil domain containing 15 (CCDC15), INO80 complex subunit B (INO80B), LOC126062579, LOC126063153, LOC126063990, LOC126063991, LOC126066513, LOC126066877, acyl-CoA wax alcohol acyltransferase 1 (AWAT1), transmembrane protein 187 (TMEM187), and LOC126069912, wherein the deleted nucleic acid sequence upstream of (a) LOC126071805 comprises SEQ ID NO:43, (b) WASHC4 comprises SEQ ID NO:44, (c) LOC126075532 comprises SEQ ID NO:45, (d) LOC126075533 comprises SEQ ID NO:46, (e) AFP comprises SEQ ID NO:47, (f) LOC126079103 comprises SEQ ID NO:48, (g) LOC126079327 comprises SEQ ID NO:49, (h) LOC126079327 comprises SEQ ID NO:50, (i) LOC126080333 comprises SEQ ID NO:51. (j) LOC126080367 comprises SEQ ID NO:52, (k) LOC126080369 comprises SEQ ID NO:53, (l) LOC126080369 comprises SEQ ID NO:54, (m) LOC126080733 comprises SEQ ID NO:55, (n) DLX6 comprises SEQ ID NO:56, (o) ECM2 comprises SEQ ID NO:57, (p) LOC126085059 comprises SEQ ID NO:58, (q) LOC126085481 comprises SEQ ID NO:59, (r) LOC126086151 comprises SEQ ID NO:60, (s) LOC126086152 comprises SEQ ID NO:61, (t) LOC126086293 comprises SEQ ID NO:62, (u) LOC126058227 comprises SEQ ID NO:63, (v) LOC126058390 comprises SEQ ID NO:64, (w) LOC126058396 comprises SEQ ID NO:65, (x) FOXH1 comprises SEQ ID NO:66, (y) LOC126060018 comprises SEQ ID NO:67, (z) LOC126060570 comprises SEQ ID NO:68, (aa) CCDC15 comprises SEQ ID NO:69, (bb) INO80B comprises SEQ ID NO:70, (cc) LOC126062579 comprises SEQ ID NO:71, (dd) LOC126063153 comprises SEQ ID NO:72, (ee) LOC126063990 comprises SEQ ID NO:73, (ff) LOC126063991 comprises SEQ ID NO:74, (gg) LOC126066513 comprises SEQ ID NO:75, (hh) LOC126066877 comprises SEQ ID NO:76, (ii) AWAT1 comprises SEQ ID NO:77, (jj) TMEM187 comprises SEQ ID NO:78, and (kk) LOC126069912 comprises SEQ ID NO: 79.

In certain embodiments, the recombinant host cells or transgenic animals comprising the isolated nucleic acids described herein further comprise a deletion of at least one nucleotide sequence upstream of a transcription start site of LOC126071805, WASHC4, LOC126075532, LOC126075533, AFP, LOC126079103, LOC126079327, LOC126079327, LOC126080333, LOC126080367, LOC126080369, LOC126080369, LOC126080733, DLX6. ECM2, LOC126085059, LOC126085481, LOC126086151, LOC126086152. LOC126086293, LOC126058227, LOC126058390, LOC126058396, FOXH1, LOC126060018, LOC126060570, CCDC15, INO80B, LOC126062579, LOC126063153, LOC126063990, LOC126063991, LOC126066513, LOC126066877, AWAT1, TMEM187, and LOC126069912, wherein the deleted nucleic acid sequence upstream of (a) LOC126071805 comprises SEQ ID NO:43, (b) WASHC4 comprises SEQ ID NO:44, (c) LOC126075532 comprises SEQ ID NO:45, (d) LOC126075533 comprises SEQ ID NO:46, (e) AFP comprises SEQ ID NO:47, (f) LOC126079103 comprises SEQ ID NO:48, (g) LOC126079327 comprises SEQ ID NO:49, (h) LOC126079327 comprises SEQ ID NO:50, (i) LOC126080333 comprises SEQ ID NO:51, (j) LOC126080367 comprises SEQ ID NO:52, (k) LOC126080369 comprises SEQ ID NO:53, (l) LOC126080369 comprises SEQ ID NO:54, (m) LOC126080733 comprises SEQ ID NO:55, (n) DLX6 comprises SEQ ID NO:56, (o) ECM2 comprises SEQ ID NO:57, (p) LOC126085059 comprises SEQ ID NO:58, (q) LOC126085481 comprises SEQ ID NO:59, (r) LOC126086151 comprises SEQ ID NO:60, (s) LOC126086152 comprises SEQ ID NO:61, (t) LOC126086293 comprises SEQ ID NO:62, (u) LOC126058227 comprises SEQ ID NO:63, (v) LOC126058390 comprises SEQ ID NO:64, (w) LOC126058396 comprises SEQ ID NO:65, (x) FOXH1 comprises SEQ ID NO:66, (y) LOC126060018 comprises SEQ ID NO:67, (z) LOC126060570 comprises SEQ ID NO:68, (aa) CCDC15 comprises SEQ ID NO:69, (bb) INO80B comprises SEQ ID NO:70, (cc) LOC126062579 comprises SEQ ID NO:71, (dd) LOC126063153 comprises SEQ ID NO:72, (ee) LOC126063990 comprises SEQ ID NO:73, (ff) LOC126063991 comprises SEQ ID NO:74, (gg) LOC126066513 comprises SEQ ID NO:75, (hh) LOC126066877 comprises SEQ ID NO:76, (ii) AWAT1 comprises SEQ ID NO:77, (jj) TMEM187 comprises SEQ ID NO:78, and (kk) LOC126069912 comprises SEQ ID NO:79.

In certain embodiments, the recombinant host cells or transgenic animals further comprise at least one woolly mammoth gene variant as described in Table 1 in WO2022/125940, which is incorporated by reference herein in its entirety. The woolly mammoth gene variants as described in WO2022/125940 are involved in a range of biological processes, including, but not limited to, regulation of cold sensitivity, regulation of heat sensitivity, regulation of intracellular pH, regulation of axonogenesis and development, tRNA, metabolic processes, cellular adhesion, tissue development and formation, microtubule-based movement of cells, negative regulation of biological processes, gene expression, cellular macromolecule metabolic processes, and the like.

The woolly mammoth gene variants described herein can be used in any combination to be expressed in any recombinant host cell or transgenic animal as described herein. In some embodiments, at least one isolated nucleic acid comprised by a recombinant host cell or transgenic animal encodes GPR98 (SEQ ID NO:1). In some embodiments, two isolated nucleic acids comprised by a recombinant host cell or transgenic animal encodes GPR98 (SEQ ID NO:1) and MACF1 (SEQ ID NO:2). In some embodiments, three isolated nucleic acids comprised by a recombinant host cell or transgenic animal encodes GPR98 (SEQ ID NO:1), MACF1 (SEQ ID NO:2), and ADARB2 (SEQ ID NO:3). In some embodiments, four isolated nucleic acids comprised by a recombinant host cell or transgenic animal encodes GPR98 (SEQ ID NO:1), MACF1 (SEQ ID NO:2), ADARB2 (SEQ ID NO:3), and CEP290 (SEQ ID NO:4). In some embodiments, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 isolated nucleic acids are comprised by the recombinant host cell to encode 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 of the woolly mammoth gene variants described herein. In some embodiments, the recombinant host cells or transgenic animals described further comprise a deletion in at least one nucleotide sequence upstream of a transcription start site of LOC126071805, WASHC4, LOC126075532, LOC126075533, AFP, LOC126079103, LOC126079327, LOC126079327, LOC126080333, LOC126080367, LOC126080369, LOC126080369, LOC126080733, DLX6, ECM2, LOC126085059, LOC126085481, LOC126086151, LOC126086152, LOC126086293, LOC126058227, LOC126058390, LOC126058396, FOXH1, LOC126060018, LOC126060570, CCDC15, INO80B, LOC126062579, LOC126063153, LOC126063990, LOC126063991, LOC126066513, LOC126066877, AWAT1. TMEM187, and LOC126069912 as described herein.

In certain embodiments, the recombinant host cell is an Asian elephant (Elephas maximus) cell, an African elephant (Loxodonta africana) cell, an African forest elephant (Loxodonta cyclotis) cell, or a Bornean elephant (Elephas maximus borneensis) cell.

In certain embodiments, the transgenic animal is an Asian elephant (Elephas maximus), an African elephant (Loxodonta africana), an African forest elephant (Loxodonta cyclotis), or a Bornean elephant (Elephas maximus borneensis).

### Cells

The woolly mammoth gene variants described herein can be expressed by any viable cell that can accept exogenous genetic material. The cell can be, for example, a prokaryotic cell or a eukaryotic cell. In some embodiments, the cell is a eukaryotic cell. The cell can be a reprogrammed cell, a non-human oocyte, a cell of a non-human embryo or a cell of a non-human blastula. In some embodiments of any of the aspects, the cell is a fibroblast cell. In some embodiments, the cell is selected from the group consisting of a nerve cell, a cartilage cell, a bone cell, a muscle cell, a bone cell, a fat cell, and an epidermal cell. In some embodiments, the cell was previously differentiated into a cell selected from the group consisting of a nerve cell, cartilage cell, bone cell, muscle cell, bone cell, fat cell, and an epidermal cell.

The scientific literature provides guidance for one of ordinary skill in the art to isolate and prepare cells as necessary for use in with the isolated nucleic acids and vectors described herein.

The cells described herein can be from any viable non-human source or organism. Usually, the organism is an animal or vertebrate such as a wild animal, zoo animal, endangered animal, rodent, domestic animal, or bird. Animals can include, as non-limiting examples, an elephant, hippopotamus, hyrax, manatee, bear, panda, feline species, e.g., tiger, lion, cheetah, bobcat, canine species, e.g., fox, wolf, avian species, e.g., ostrich, emu, penguin, pigeon, and fish, e.g., trout, catfish, and salmon. In some embodiments, the cell described herein is from a mammal. Non-limiting examples of organisms from which cells can be derived include: elephants (e.g., Loxodonta africana, Elephas maximus, Loxodonta cyclotis, Elephas maximus borneensis); hyrax (e.g., Dendrohyrax arboreus, Dendrohyrax dorsalis, Heterohyrax brucei, Procavia capensis); aardvark (e.g., Oryceteropus afer); shrew (e.g., Suncus etruscus, Blarina brevicauda, Neomys fodiens), and manatees (Trichechus inunguis, Trichechus manatus, Trichechus manatus latirostris, Trichechus manatus manatus, Trichechus senegalensis).

In certain embodiments, a cell useful in the methods and compositions described herein is an elephant cell. In some embodiments, the cell is an elephant fibroblast cell. In some embodiments, the cell is an elephant stem cell. In some embodiments, the cell described herein is an elephant somatic cell reprogrammed to a stem cell or stem cell-like phenotype having stem cell-like morphology and/or expressing at least one stem cell marker described herein.

The cells described herein can be from any tissue isolated from an organism by methods known in the art. For example, placental tissue can be isolated from a given organism (e.g., an elephant), after full term delivery of young, and subsequently processed for cellular isolation and/or culture by methods known in the art. Additional exemplary cell types that can be used for the compositions and methods described herein include but are not limited to fibroblasts, skin cells, blood cells (e.g., leukocytes, monocytes, dendritic cells), stem cells, hematopoietic cells, liver cells, vascular cells, muscle cells, pancreatic cells, neural cells, ocular or retinal cells, epithelial or endothelial cells, lung cells, cardiac cells, intestinal cells, diaphragmatic cells, renal (i.e., kidney) cells, bone marrow cells, or any one or more selected tissues or cells of an organism for which genetic modification or gene editing to express a woolly mammoth gene is contemplated.

In certain embodiments, the isolated nucleic acids and vectors described herein are used in stem cells. Stem cells are cells that retain the ability to renew themselves through mitotic cell division and can differentiate into more specialized cell types. Three broad types of mammalian stem cells include: embryonic stem (ES) cells that are found in blastocysts, induced pluripotent stem cells (iPSCs) that are reprogrammed from somatic cells, and adult stem cells that are found in adult tissues. Other sources of stem cells can include, for example, amnion-derived or placental-derived stem cells. Pluripotent stem cells can differentiate into cells derived from any of the three germ layers.

In certain embodiments, the recombinant host cell is a stem cell. The stem cell can, for example, be selected from an induced stem cell, embryonic stem (ES) cell, or a mesenchymal stem cell (MSC). In certain embodiments, the recombinant host cell is a reprogrammed cell. In certain embodiments, the recombinant host cell is a fibroblast cell or a mesenchymal cell. In certain embodiments, the recombinant host cell is selected from the group consisting of a nerve cell, a cartilage cell, a bone cell, a muscle cell, a bone cell, a fat cell, and an epidermal cell.

In certain embodiments, the recombinant host cell fails to express an endogenous homologue of at least one of GPR98, MACF1, ADARB2, CEP290, KRT4, LOC126076011, NCKAP5, LAMB4, NPC1L1, ADGRD2, NINJ1, AHNAK2, CATSPERB, PCNXL4, SYNE2. NLRP12, LOC126086768, WDR90. BRCA2. PRKDC, VPS13B. PSAP, SEC31B. KRT28, KRT35, KRT40, MYH4, PIRT, PKD1L2, RP1L1, CXorf58, LOC126068772, LOC126069872, THRAP3, CENPC1, DSPP, FGF5, CATSPERG, MYH1, MYH13, ATRIP, and TGM3. The recombinant host cell can, for example, fail to express the endogenous homologue of 2, 3.4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 of GPR98, MACF1, ADARB2, CEP290, KRT4, LOC126076011, NCKAP5, LAMB4, NPC1L1, ADGRD2, NINJ1, AHNAK2, CATSPERB, PCNXL4, SYNE2, NLRP12, LOC126086768, WDR90, BRCA2, PRKDC, VPS13B, PSAP, SEC31B, KRT28, KRT35, KRT40, MYH4, PIRT, PKDIL2, RP1L1, CXorf58, LOC126068772, LOC126069872, THRAP3, CENPC1, DSPP, FGF5, CATSPERG, MYH1, MYH13, ATRIP, and TGM3.

In certain embodiments, the recombinant host cell is an elephant cell. The elephant cell can, for example, be selected from an Asian elephant cell (Elephas maximus), an African elephant cell (Loxodonta africana), an African forest elephant cell (Loxodonta cyclotis), and a Bornean elephant cell (Elephas maximus borneensis).

Also provided are transgenic animals comprising a recombinant host cell as described herein. In certain embodiments, the transgenic animal is an Asian elephant (Elephas maximus), an African elephant (Loxodonta africana), an African forest elephant (Loxodonta cyclotis), and a Bornean elephant (Elephas maximus borneensis).

### Transgenic Animals

Also provided are transgenic animals comprising at least one woolly mammoth (Mammuthus primigenius) gene variant. The at least one woolly mammoth (Mammuthus primigenius) gene variant can, for example, be selected from the group consisting of GPR98 (SEQ ID NO:1). MACF1 (SEQ ID NO:2), ADARB2 (SEQ ID NO:3), CEP290 (SEQ ID NO:4), KRT4 (SEQ ID NO:5), LOC126076011 (SEQ ID NO:6), NCKAP5 (SEQ ID NO:7), LAMB4 (SEQ ID NO:8), NPC1L1 (SEQ ID NO:9), ADGRD2 (SEQ ID NO:10), NINJ1 (SEQ ID NO:11), AHNAK2 (SEQ ID NO:12), CATSPERB (SEQ ID NO: 13), PCNXL4 (SEQ ID NO:14), SYNE2 (SEQ ID NO:15), NLRP12 (SEQ ID NO:16), LOC126086768 (SEQ ID NO:17), WDR90 (SEQ ID NO:18), BRCA2 (SEQ ID NO:19), PRKDC (SEQ ID NO:20), VPS13B (SEQ ID NO:21), PSAP (SEQ ID NO:22), SEC31B (SEQ ID NO:23), KRT28 (SEQ ID NO:24), KRT35 (SEQ ID NO:25), KRT40 (SEQ ID NO:26), MYH4 (SEQ ID NO:27), PIRT (SEQ ID NO:28), PKD1L2 (SEQ ID NO:29), RP1L1 (SEQ ID NO:30), CXorf58 (SEQ ID NO:31), LOC126068772 (SEQ ID NO:32), LOC126069872 (SEQ ID NO:33), THRAP3 (SEQ ID NO:34), CENPC1 (SEQ ID NO:35), DSPP (SEQ ID NO:36), FGF5 (SEQ ID NO:37), CATSPERG (SEQ ID NO:38), MYH1 (SEQ ID NO:39), MYH13 (SEQ ID NO:40), ATRIP (SEQ ID NO:41), and TGM3 (SEQ ID NO:42).

In certain embodiments, the transgenic animal comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 mammoth gene variants.

In certain embodiments, the transgenic animal fails to express an endogenous homologue of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 of GPR98 (SEQ ID NO:1), MACF1 (SEQ ID NO:2), ADARB2 (SEQ ID NO:3), CEP290 (SEQ ID NO:4), KRT4 (SEQ ID NO:5), LOC126076011 (SEQ ID NO:6), NCKAP5 (SEQ ID NO:7), LAMB4 (SEQ ID NO:8), NPC1L1 (SEQ ID NO:9), ADGRD2 (SEQ ID NO:10). NINJ1 (SEQ ID NO: 1l). AHNAK2 (SEQ ID NO:12), CATSPERB (SEQ ID NO:13), PCNXL4 (SEQ ID NO:14), SYNE2 (SEQ ID NO:15), NLRP12 (SEQ ID NO:16), LOC126086768 (SEQ ID NO:17), WDR90 (SEQ ID NO:18), BRCA2 (SEQ ID NO:19), PRKDC (SEQ ID NO:20), VPS13B (SEQ ID NO:21), PSAP (SEQ ID NO:22), SEC31B (SEQ ID NO:23), KRT28 (SEQ ID NO:24), KRT35 (SEQ ID NO:25), KRT40 (SEQ ID NO:26), MYH4 (SEQ ID NO:27), PIRT (SEQ ID NO:28), PKD1L2 (SEQ ID NO:29), RP1L1 (SEQ ID NO:30), CXorf58 (SEQ ID NO:31), LOC126068772 (SEQ ID NO:32), LOC126069872 (SEQ ID NO:33), THRAP3 (SEQ ID NO:34), CENPC1 (SEQ ID NO:35), DSPP (SEQ ID NO:36), FGF5 (SEQ ID NO:37), CATSPERG (SEQ ID NO:38), MYH1 (SEQ ID NO:39), MYH13 (SEQ ID NO:40), ATRIP (SEQ ID NO:41), and TGM3 (SEQ ID NO:42).

In certain embodiments, the transgenic animal further comprises a deletion of at least one nucleotide sequence upstream of a transcription start site of LOC126071805, WASHC4, LOC126075532, LOC126075533, AFP, LOC126079103, LOC126079327, LOC126079327, LOC126080333, LOC126080367, LOC126080369, LOC126080369, LOC126080733, DLX6, ECM2, LOC126085059, LOC126085481, LOC126086151, LOC126086152, LOC126086293, LOC126058227, LOC126058390, LOC126058396, FOXH1, LOC126060018, LOC126060570. CCDC15, INO80B, LOC126062579, LOC126063153, LOC126063990, LOC126063991, LOC126066513, LOC126066877, AWAT1, TMEM187, and LOC126069912, wherein the deleted nucleic acid sequence upstream of (a) LOC126071805 comprises SEQ ID NO:43, (b) WASHC4 comprises SEQ ID NO:44, (c) LOC126075532 comprises SEQ ID NO:45, (d) LOC126075533 comprises SEQ ID NO:46, (e) AFP comprises SEQ ID NO:47, (f) LOC126079103 comprises SEQ ID NO:48, (g) LOC126079327 comprises SEQ ID NO:49, (h) LOC126079327 comprises SEQ ID NO:50, (i) LOC126080333 comprises SEQ ID NO:51, (j) LOC126080367 comprises SEQ ID NO:52, (k) LOC126080369 comprises SEQ ID NO:53, (l) LOC126080369 comprises SEQ ID NO:54, (m) LOC126080733 comprises SEQ ID NO:55, (n) DLX6 comprises SEQ ID NO:56, (o) ECM2 comprises SEQ ID NO:57, (p) LOC126085059 comprises SEQ ID NO:58, (q) LOC126085481 comprises SEQ ID NO:59, (r) LOC126086151 comprises SEQ ID NO:60, (s) LOC126086152 comprises SEQ ID NO:61, (t) LOC126086293 comprises SEQ ID NO:62, (u) LOC126058227 comprises SEQ ID NO:63, (v) LOC126058390 comprises SEQ ID NO:64, (w) LOC126058396 comprises SEQ ID NO:65, (x) FOXH1 comprises SEQ ID NO:66, (y) LOC126060018 comprises SEQ ID NO:67, (z) LOC126060570 comprises SEQ ID NO:68, (aa) CCDC15 comprises SEQ ID NO:69, (bb) INO80B comprises SEQ ID NO:70, (cc) LOC126062579 comprises SEQ ID NO:71, (dd) LOC126063153 comprises SEQ ID NO:72. (ee) LOC126063990 comprises SEQ ID NO:73, (ff) LOC126063991 comprises SEQ ID NO:74, (gg) LOC126066513 comprises SEQ ID NO:75, (hh) LOC126066877 comprises SEQ ID NO:76, (ii) AWAT1 comprises SEQ ID NO:77, (jj) TMEM187 comprises SEQ ID NO:78, and (kk) LOC126069912 comprises SEQ ID NO:79.

In certain embodiments, the transgenic animal is an Asian elephant (Elephas maximus), an African elephant (Loxodonta africana), an African forest elephant (Loxodonta cyclotis), or a Bornean elephant (Elephas maximus borneensis).

Methods for Introducing Woolly Mammoth Gene Variants or Deletions of Regulatory Elements into a Cell

In certain embodiments of any of the aspects, the cell compositions described herein express a polypeptide encoded by the at least one isolated nucleic acid sequence having a woolly mammoth gene variant nucleotide sequence (including, but not limited to the exogenous woolly mammoth gene variants, as described above, and in Table 1 of WO2022/125940, which is incorporated by reference herein in its entirety).

The cells described herein can be transfected, contacted with, or administered an exogenous woolly mammoth gene encoded by the isolated nucleic acids described herein by methods known in the art.

In some embodiments, the at least one nucleic acid sequence encoding a woolly mammoth gene is delivered via a vector.

A vector is a nucleic acid construct designed for delivery to a host cell or for transfer of genetic material between different host cells. As used herein, a vector can be viral or non-viral. The term “vector” encompasses any genetic element that is capable of replication when associated with the proper control elements and that can transfer genetic material to cells. A vector can include, but is not limited to, a cloning vector, an expression vector, a plasmid, phage, transposon, cosmid, artificial chromosome, virus, virion, etc.

In some embodiments of any of the aspects, the vector is selected from the group consisting of: a plasmid, a cosmid and a viral vector.

An expression vector is a vector that directs expression of an RNA or polypeptide (e.g., a woolly mammoth polypeptide) from nucleic acid sequences contained therein linked to transcriptional regulatory sequences on the vector. The sequences expressed will often, but not necessarily, be heterologous to the cell; a woolly mammoth gene introduced to a viable cell is heterologous to the cell. An expression vector may comprise additional elements, for example, the expression vector may have two replication systems, thus allowing it to be maintained in two organisms, for example in animal cells for expression and in a prokaryotic host for cloning and amplification. “Expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene.

In some embodiments, a vector is capable of driving expression of one or more sequences in a mammalian cell; i.e., the vector is a mammalian expression vector. Examples of mammalian expression vectors include pCDM8 (Seed, 1987. Nature 329: 840) and pMT2PC (Kaufman, et al., 1987. EMBO J. 6: 187-195), When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

### Methods of Inhibiting or Editing the Expression of an Endogenous Gene

In some embodiments of any the aspects, the cell described herein does not express an endogenous homologue of the at least one woolly mammoth gene variant described herein. In another embodiment of any of the aspects, the cell is edited to inhibit expression of an endogenous homologue of the at least one woolly mammoth gene variant. In another embodiment of any of the aspects, the cell is edited to alter the regulatory and/or coding region to incorporate substitutions so that the endogenous homologue resembles the at least one gene woolly mammoth gene variant.

In another embodiment of any of the aspects, the non-woolly mammoth homologue of the exogenous nucleic acid sequence has been deleted or inactivated.

It is contemplated herein that when one or more woolly mammoth gene variants are delivered to the host cell(s) it can be advantageous to modify the endogenous non-woolly mammoth homologue of the one or more genes to render the endogenous gene or genes non-functional. It is further contemplated herein that if two or more woolly mammoth genes are delivered to the host cell, one or both of the endogenous host cell genes would be altered. Thus, in this context, the host cell can comprise at least one non-functional endogenous homologue to the corresponding woolly mammoth gene.

In the context of elephant cells, the elephant homologue(s) of the one or more woolly mammoth genes to be expressed would be altered, deleted, or inhibited such that only the one or more woolly mammoth genes is/are expressed by the cell. This can be achieved, for example, by standard gene editing of target sequences. It is also contemplated that rather than simply inactivating the endogenous gene, wholesale replacement of the endogenous gene, e.g., via homologous recombination, or via selective editing of the non-mammoth homologue gene(s) to encode and express the mammoth variant gene sequence(s) could also be performed.

The target sequence can be determined by methods known in the art. For example, sequence alignment tools can be used to compare the woolly mammoth nucleic acid sequences to those in the host organism, e.g., using NCBI Basic Local Alignment Sequence Tool (BLAST), OrthoMaM, Ensembl and/or Megalign (DNASTAR) software. Those skilled in the art can determine appropriate parameters for aligning sequences, including any algorithms needed to achieve maximal alignment over the full length of the sequences being compared.

Methods of inhibiting gene function in a host cell are known in the art. Non-limiting examples of gene knockdown, inhibition, and alteration include, e.g., gene editing enzymes, Transcription Activator-Like Effectors Nucleases (TALENS), inhibitory nucleic acids, and the like. Exemplary embodiments of types of inhibitory nucleic acids can include, e.g., siRNA, shRNA, miRNA, and/or a miRNA, which are known in the art. One of ordinary skill in the art can design and test an inhibitory agent that targets the endogenous homologue of a thylacine gene variant described herein.

Methods of preparing and delivering gene editing systems are described, e.g., in WO2015/013583 A2; U.S. Pat. No. 10,640,789 B2; US Publication No. US2019/0367948 A1; US Publication No. 2017/0266320 A1; US Publication No. 2018/0171361 A1; US Publication No. 2016/017546 2 A1; and US Publication No. 2018/0195089 A1, the contents of each of which are incorporated herein by reference in their entirety.

### Embodiments

The invention provides also the following non-limiting embodiments.

Embodiment 1 is an isolated nucleic acid sequence comprising a nucleotide sequence encoding an amino acid sequence having at least 80%, at least 85%, at least 90%, at least 95%, at least 96%, at least 97%, at least 98%, or at least 99% identity to at least one of G protein-coupled receptor 98 (GPR98) (SEQ ID NO: 1), microtubule actin crosslinking factor 1 (MACF1) (SEQ ID NO:2), adenosine deaminase RNA specific B2 (ADARB2) (SEQ ID NO:3), centrosomal protein 290 (CEP290) (SEQ ID NO:4), keratin 4 (KRT4) (SEQ ID NO:5), LOC126076011 (SEQ ID NO:6), NCK associated protein 5 (NCKAP5) (SEQ ID NO:7), laminin subunit beta 4 (LAMB4) (SEQ ID NO:8). Niemann-Pick C1-like 1 (NPC1L1) (SEQ ID NO:9), adhesion G protein-coupled receptor D2 (ADGRD2) (SEQ ID NO:10), ninjurin 1 (NINJ1) (SEQ ID NO:11), AH-NAK nucleoprotein 2 (AHNAK2) (SEQ ID NO: 12), cation channel sperm associated auxiliary subunit beta (CATSPERB) (SEQ ID NO:13), pecanex-like 4 (PCNXL4) (SEQ ID NO: 14), spectrin repeat containing nuclear envelope protein 2 (SYNE2) (SEQ ID NO:15), NLR family pyrin domain containing 12 (NLRP12) (SEQ ID NO:16), LOC126086768 (SEQ ID NO:17), WD repeat domain 90 (WDR90) (SEQ ID NO:18), breast cancer 2 (BRCA2) (SEQ ID NO:19), protein kinase, DNA-activated catalytic subunit (PRKDC) (SEQ ID NO:20), vacuolar protein sorting 13 homolog B (VPS13B) (SEQ ID NO:21), prosaposin (PSAP) (SEQ ID NO:22), SEC31 homolog B (SEC31B) (SEQ ID NO:23), keratin 28 (KRT28) (SEQ ID NO:24), keratin 35 (KRT35) (SEQ ID NO:25), keratin 40 (KRT40) (SEQ ID NO:26), myosin heavy chain 4 (MYH4) (SEQ ID NO:27), phosphoinositide interacting regulator of TRP (PIRT) (SEQ ID NO:28), polycystin 1 like 2 (PKD1L2) (SEQ ID NO:29), retinitis pigmentosa 1-like protein (RP1L1) (SEQ ID NO:30), chromosome X open reading frame 58 (CXorf58) (SEQ ID NO:31), LOC126068772 (SEQ ID NO:32), LOC126069872 (SEQ ID NO:33), thyroid hormone receptor associated protein 3 (THRAP3) (SEQ ID NO:34), centromere protein C 1 (CENPC1) (SEQ ID NO:35), dentinogenesis and dentin sialophosphoprotein (DSPP) (SEQ ID NO:36), fibroblast growth factor 5 (FGF5) (SEQ ID NO:37), cation channel sperm associated auxiliary subunit gamma (CATSPERG) (SEQ ID NO:38), myosin heavy chain 1 (MYH1) (SEQ ID NO:39), myosin heavy chain 13 (MYH13) (SEQ ID NO:40), ATR interacting protein (ATRIP) (SEQ ID NO:41), and transglutaminase 3 (TGM3) (SEQ ID NO:42).

Embodiment 2 is the isolated nucleic acid sequence of embodiment 1, wherein the nucleotide sequence encodes an amino acid of at least one of GPR98 (SEQ ID NO:1), MACF1 (SEQ ID NO:2), ADARB2 (SEQ ID NO:3), CEP290 (SEQ ID NO:4), KRT4 (SEQ ID NO:5), LOC126076011 (SEQ ID NO:6), NCKAP5 (SEQ ID NO:7), LAMB4 (SEQ ID NO:8), NPC1L1 (SEQ ID NO:9), ADGRD2 (SEQ ID NO:10), NINJ1 (SEQ ID NO:11), AHNAK2 (SEQ ID NO:12), CATSPERB (SEQ ID NO:13), PCNXL4 (SEQ ID NO:14), SYNE2 (SEQ ID NO:15), NLRP12 (SEQ ID NO:16), LOC126086768 (SEQ ID NO: 17), WDR90 (SEQ ID NO:18), BRCA2 (SEQ ID NO:19), PRKDC (SEQ ID NO:20), VPS13B (SEQ ID NO:21), PSAP (SEQ ID NO:22), SEC31B (SEQ ID NO:23), KRT28 (SEQ ID NO:24), KRT35 (SEQ ID NO:25), KRT40 (SEQ ID NO:26), MYH4 (SEQ ID NO:27), PIRT (SEQ ID NO:28), PKD1L2 (SEQ ID NO:29), RP1L1 (SEQ ID NO:30), CXorf58 (SEQ ID NO:31), LOC126068772 (SEQ ID NO:32), LOC126069872 (SEQ ID NO:33), THRAP3 (SEQ ID NO:34), CENPC1 (SEQ ID NO:35), DSPP (SEQ ID NO:36), FGF5 (SEQ ID NO:37), CATSPERG (SEQ ID NO:38), MYH1 (SEQ ID NO:39), MYH13 (SEQ ID NO:40), ATRIP (SEQ ID NO:41), and TGM3 (SEQ ID NO:42).

Embodiment 3 is an isolated vector comprising the isolated nucleic acid sequence of embodiment 1 or 2.

Embodiment 4 is a recombinant host cell comprising at least one isolated nucleic acid sequence of embodiment 1 or 2.

Embodiment 5 is a recombinant host cell comprising a nucleotide sequence having at least 80%, at least 85%, at least 90%, at least 95%, at least 96%, at least 97%, at least 98%, or at least 99% identity to at least one of GPR98 (SEQ ID NO: 1), MACF1 (SEQ ID NO:2), ADARB2 (SEQ ID NO:3), CEP290 (SEQ ID NO:4), KRT4 (SEQ ID NO:5), LOC126076011 (SEQ ID NO:6), NCKAP5 (SEQ ID NO:7), LAMB4 (SEQ ID NO:8), NPC1L1 (SEQ ID NO:9), ADGRD2 (SEQ ID NO: 10), NINJ1 (SEQ ID NO: 11). AHNAK2 (SEQ ID NO:12), CATSPERB (SEQ ID NO: 13), PCNXL4 (SEQ ID NO:14). SYNE2 (SEQ ID NO: 15), NLRP12 (SEQ ID NO:16), LOC126086768 (SEQ ID NO:17), WDR90 (SEQ ID NO:18), BRCA2 (SEQ ID NO:19), PRKDC (SEQ ID NO:20), VPS13B (SEQ ID NO:21), PSAP (SEQ ID NO:22), SEC31B (SEQ ID NO:23), KRT28 (SEQ ID NO:24), KRT35 (SEQ ID NO:25), KRT40 (SEQ ID NO:26), MYH4 (SEQ ID NO:27), PIRT (SEQ ID NO:28), PKD1L2 (SEQ ID NO:29), RP1L1 (SEQ ID NO:30), CXorf58 (SEQ ID NO:31), LOC126068772 (SEQ ID NO:32), LOC126069872 (SEQ ID NO:33), THRAP3 (SEQ ID NO:34), CENPC1 (SEQ ID NO:35), DSPP (SEQ ID NO:36), FGF5 (SEQ ID NO:37), CATSPERG (SEQ ID NO:38), MYH1 (SEQ ID NO:39), MYH13 (SEQ ID NO:40), ATRIP (SEQ ID NO:41), and TGM3 (SEQ ID NO:42).

Embodiment 6 is the recombinant host cell of embodiment 5, wherein the host cell comprises a nucleotide sequence having at least one of GPR98 (SEQ ID NO:1), MACF1 (SEQ ID NO:2), ADARB2 (SEQ ID NO:3), CEP290 (SEQ ID NO:4), KRT4 (SEQ ID NO:5), LOC126076011 (SEQ ID NO:6), NCKAP5 (SEQ ID NO:7), LAMB4 (SEQ ID NO:8), NPC1L1 (SEQ ID NO:9), ADGRD2 (SEQ ID NO:10), NINJ1 (SEQ ID NO:11), AHNAK2 (SEQ ID NO:12), CATSPERB (SEQ ID NO:13), PCNXL4 (SEQ ID NO:14), SYNE2 (SEQ ID NO:15), NLRP12 (SEQ ID NO: 16), LOC126086768 (SEQ ID NO: 17), WDR90 (SEQ ID NO:18), BRCA2 (SEQ ID NO:19), PRKDC (SEQ ID NO:20), VPS13B (SEQ ID NO:21), PSAP (SEQ ID NO:22), SEC31B (SEQ ID NO:23), KRT28 (SEQ ID NO:24), KRT35 (SEQ ID NO:25), KRT40 (SEQ ID NO:26), MYH4 (SEQ ID NO:27), PIRT (SEQ ID NO:28), PKD1L2 (SEQ ID NO:29), RP1L1 (SEQ ID NO:30), CXorf58 (SEQ ID NO:31), LOC126068772 (SEQ ID NO:32), LOC126069872 (SEQ ID NO:33), THRAP3 (SEQ ID NO:34), CENPC1 (SEQ ID NO:35), DSPP (SEQ ID NO:36), FGF5 (SEQ ID NO:37), CATSPERG (SEQ ID NO:38), MYH1 (SEQ ID NO:39), MYH13 (SEQ ID NO:40), ATRIP (SEQ ID NO:41), and TGM3 (SEQ ID NO:42).

Embodiment 7 is the recombinant host cell of any one of embodiments 4-6, wherein the recombinant host cell comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 isolated nucleic acids of claim 1 or 2.

Embodiment 8 is a recombinant host cell comprising a deletion of at least one nucleotide sequence upstream of a transcription start site of LOC126071805, WASH complex subunit 4 (WASHC4), LOC126075532, LOC126075533, alpha-fetoprotein (AFP), LOC126079103, LOC126079327, LOC126079327, LOC126080333, LOC126080367, LOC126080369, LOC126080369, LOC126080733, distal-less homeobox 6 (DLX6), extracellular matrix protein 2 (ECM2), LOC126085059, LOC126085481, LOC126086151, LOC126086152, LOC126086293, LOC126058227, LOC126058390, LOC126058396, forkhead box H1 (FOXH1), LOC126060018, LOC126060570, coiled-coil domain containing 15 (CCDC15), INO80 complex subunit B (INO80B), LOC126062579, LOC126063153, LOC126063990, LOC126063991, LOC126066513, LOC126066877, acyl-CoA wax alcohol acyltransferase 1 (AWAT1), transmembrane protein 187 (TMEM187), and LOC126069912, wherein the deleted nucleic acid sequence upstream of:
(a) LOC126071805 comprises SEQ ID NO:43,
(b) WASHC4 comprises SEQ ID NO:44,
(c) LOC126075532 comprises SEQ ID NO:45,
(d) LOC126075533 comprises SEQ ID NO:46,
(e) AFP comprises SEQ ID NO:47,
(f) LOC126079103 comprises SEQ ID NO:48,
(g) LOC126079327 comprises SEQ ID NO:49,
(h) LOC126079327 comprises SEQ ID NO:50,
(i) LOC126080333 comprises SEQ ID NO:51,
(j) LOC126080367 comprises SEQ ID NO:52,
(k) LOC126080369 comprises SEQ ID NO:53,
(l) LOC126080369 comprises SEQ ID NO:54,
(m) LOC126080733 comprises SEQ ID NO:55,
(n) DLX6 comprises SEQ ID NO:56,
(o) ECM2 comprises SEQ ID NO:57,
(p) LOC126085059 comprises SEQ ID NO:58,
(q) LOC126085481 comprises SEQ ID NO:59,
(r) LOC126086151 comprises SEQ ID NO:60,
(s) LOC126086152 comprises SEQ ID NO:61,
(t) LOC126086293 comprises SEQ ID NO:62,
(u) LOC126058227 comprises SEQ ID NO: 63,
(v) LOC126058390 comprises SEQ ID NO: 64,
(w) LOC126058396 comprises SEQ ID NO:65,
(x) FOXH1 comprises SEQ ID NO:66,
(y) LOC126060018 comprises SEQ ID NO:67,
(z) LOC126060570 comprises SEQ ID NO:68,
(aa) CCDC15 comprises SEQ ID NO:69.
(bb) INO80B comprises SEQ ID NO:70,
(cc) LOC126062579 comprises SEQ ID NO:71,
(dd) LOC126063153 comprises SEQ ID NO:72,
(ee) LOC126063990 comprises SEQ ID NO:73,
(ff) LOC126063991 comprises SEQ ID NO:74,
(gg) LOC126066513 comprises SEQ ID NO:75,
(hh) LOC126066877 comprises SEQ ID NO:76,
(ii) AWAT1 comprises SEQ ID NO:77,
(jj) TMEM187 comprises SEQ ID NO: 78, and
(kk) LOC126069912 comprises SEQ ID NO: 79.

Embodiment 9 is the recombinant host cell of embodiment 7, further comprising a deletion of at least one nucleotide sequence upstream of a transcription start site of LOC126071805, WASHC4, LOC126075532, LOC126075533, AFP, LOC126079103, LOC126079327, LOC126079327, LOC126080333, LOC126080367, LOC126080369, LOC126080369, LOC126080733, DLX6, ECM2, LOC126085059, LOC126085481, LOC126086151, LOC126086152, LOC126086293, LOC126058227, LOC126058390, LOC126058396, FOXH1, LOC126060018, LOC126060570, CCDC15, INO80B, LOC126062579, LOC126063153, LOC126063990, LOC126063991, LOC126066513, LOC126066877, AWAT1, TMEM187, and LOC126069912, wherein the deleted nucleic acid sequence upstream of:
(a) LOC126071805 comprises SEQ ID NO:43,
(b) WASHC4 comprises SEQ ID NO:44,
(c) LOC126075532 comprises SEQ ID NO:45,
(d) LOC126075533 comprises SEQ ID NO:46,
(e) AFP comprises SEQ ID NO:47,
(f) LOC126079103 comprises SEQ ID NO:48,
(g) LOC126079327 comprises SEQ ID NO:49,
(h) LOC126079327 comprises SEQ ID NO:50,
(i) LOC126080333 comprises SEQ ID NO:51,
(j) LOC126080367 comprises SEQ ID NO:52,
(k) LOC126080369 comprises SEQ ID NO:53,
(l) LOC126080369 comprises SEQ ID NO:54,
(m) LOC126080733 comprises SEQ ID NO:55,
(n) DLX6 comprises SEQ ID NO:56,
(o) ECM2 comprises SEQ ID NO:57,
(p) LOC126085059 comprises SEQ ID NO:58,
(q) LOC126085481 comprises SEQ ID NO:59,
(r) LOC126086151 comprises SEQ ID NO:60,
(s) LOC126086152 comprises SEQ ID NO:61,
(t) LOC126086293 comprises SEQ ID NO:62,
(u) LOC126058227 comprises SEQ ID NO:63,
(v) LOC126058390 comprises SEQ ID NO:64,
(w) LOC126058396 comprises SEQ ID NO:65,
(x) FOXH1 comprises SEQ ID NO:66,
(y) LOC126060018 comprises SEQ ID NO:67,
(z) LOC126060570 comprises SEQ ID NO:68,
(aa) CCDC15 comprises SEQ ID NO:69,
(bb) INO80B comprises SEQ ID NO:70,
(cc) LOC126062579 comprises SEQ ID NO:71,
(dd) LOC126063153 comprises SEQ ID NO: 72,
(ee) LOC126063990 comprises SEQ ID NO:73,
(ff) LOC126063991 comprises SEQ ID NO:74,
(gg) LOC126066513 comprises SEQ ID NO:75,
(hh) LOC126066877 comprises SEQ ID NO:76,
(ii) AWAT1 comprises SEQ ID NO:77,
(jj) TMEM187 comprises SEQ ID NO:78, and
(kk) LOC126069912 comprises SEQ ID NO:79.

Embodiment 10 is the recombinant host cell of any one of embodiments 4-9, wherein the recombinant host cell is a stem cell.

Embodiment 11 is the recombinant host cell of embodiment 10, wherein the stem cell is selected from an induced stem cell, embryonic stem (ES) cell, or a mesenchymal stem cell (MSC).

Embodiment 12 is the recombinant host cell of any one of embodiments 4-9, wherein the recombinant host cell is a reprogrammed cell.

Embodiment 13 is the recombinant host cell of any one of embodiments 4-9, wherein the recombinant host cell is a fibroblast cell or a mesenchymal cell.

Embodiment 14 is the recombinant host cell or any one of embodiments 4-9, wherein the recombinant host cell is selected from the group consisting of a nerve cell, a cartilage cell, a bone cell, a muscle cell, a bone cell, a fat cell, and an epidermal cell.

Embodiment 15 is the recombinant host cell of any one of embodiments 4-14, wherein the recombinant host cell fails to express an endogenous homologue of at least one of GPR98, MACF1, ADARB2, CEP290, KRT4, LOC126076011, NCKAP5, LAMB4, NPC1L1, ADGRD2, NINJ1, AHNAK2, CATSPERB, PCNXL4, SYNE2, NLRP12, LOC126086768, WDR90, BRCA2, PRKDC, VPS13B, PSAP, SEC31B, KRT28, KRT35, KRT40, MYH4, PIRT, PKD1L2, RP1L1, CXorf58, LOC126068772, LOC126069872, THRAP3, CENPC1, DSPP, FGF5, CATSPERG, MYH1, MYH13, ATRIP, and TGM3.

Embodiment 16 is the recombinant host cell of embodiment 15, wherein the recombinant host cell fails to express the endogenous homologue of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 of GPR98, MACF1, ADARB2, CEP290, KRT4, LOC126076011, NCKAP5, LAMB4, NPC1L1, ADGRD2, NINJ1, AHNAK2, CATSPERB, PCNXL4, SYNE2, NLRP12, LOC126086768, WDR90, BRCA2, PRKDC, VPS13B, PSAP, SEC31B, KRT28, KRT35, KRT40, MYH4, PIRT, PKD1L2, RP1L1, CXorf58, LOC126068772, LOC126069872. THRAP3, CENPC1, DSPP, FGF5, CATSPERG, MYH1, MYH13, ATRIP, and TGM3.

Embodiment 17 is the recombinant host cell of any one of embodiments 3-16, wherein the recombinant host cell is an elephant cell.

Embodiment 18 is the recombinant host cell of embodiment 17, wherein the elephant cell is selected from an Asian elephant cell (Elephas maximus), an African elephant cell (Loxodonta africana), an African forest elephant cell (Loxodonta cyclotis), and a Bornean elephant cell (Elephas maximus borneensis).

Embodiment 19 is a transgenic animal comprising the recombinant host cell of any one of embodiments 3-18.

Embodiment 20 is the transgenic animal of embodiment 19, wherein the transgenic animal is an Asian elephant (Elephas maximus), an African elephant (Loxodonta africana), an African forest elephant (Loxodonta cyclotis), and a Bornean elephant (Elephas maximus borneensis).

Embodiment 21 is a transgenic animal comprising at least one woolly mammoth (Mammuthus primigenius) gene variant.

Embodiment 22 is the transgenic animal of embodiment 21, wherein the at least one woolly mammoth (Mammuthus primigenius) gene variant comprises a nucleotide sequence encoding an amino acid sequence having at least 80%, at least 85%, at least 90%, at least 95%, at least 96%, at least 97%, at least 98%, or at least 99% identity to at least one of GPR98 (SEQ ID NO:1), MACF1 (SEQ ID NO:2), ADARB2 (SEQ ID NO:3), CEP290 (SEQ ID NO:4), KRT4 (SEQ ID NO:5), LOC126076011 (SEQ ID NO:6), NCKAP5 (SEQ ID NO:7), LAMB4 (SEQ ID NO:8), NPC1L1 (SEQ ID NO:9). ADGRD2 (SEQ ID NO:10). NINJ1 (SEQ ID NO: 11). AHNAK2 (SEQ ID NO: 12), CATSPERB (SEQ ID NO:13), PCNXL4 (SEQ ID NO:14), SYNE2 (SEQ ID NO:15), NLRP12 (SEQ ID NO:16), LOC126086768 (SEQ ID NO:17), WDR90 (SEQ ID NO:18), BRCA2 (SEQ ID NO:19). PRKDC (SEQ ID NO:20), VPS13B (SEQ ID NO:21), PSAP (SEQ ID NO:22), SEC31B (SEQ ID NO:23), KRT28 (SEQ ID NO:24), KRT35 (SEQ ID NO:25), KRT40 (SEQ ID NO:26), MYH4 (SEQ ID NO:27), PIRT (SEQ ID NO:28), PKD1L2 (SEQ ID NO:29), RP1L1 (SEQ ID NO:30), CXorf58 (SEQ ID NO:31), LOC126068772 (SEQ ID NO:32), LOC126069872 (SEQ ID NO:33). THRAP3 (SEQ ID NO:34), CENPC1 (SEQ ID NO:35), DSPP (SEQ ID NO:36), FGF5 (SEQ ID NO:37), CATSPERG (SEQ ID NO:38), MYH1 (SEQ ID NO:39), MYH13 (SEQ ID NO:40), ATRIP (SEQ ID NO:41), and TGM3 (SEQ ID NO:42).

Embodiment 23 is the transgenic animal of embodiment 22, wherein the at least one woolly mammoth (Mammuthus primigenius) gene variant comprises at least one of GPR98 (SEQ ID NO:1), MACF1 (SEQ ID NO:2), ADARB2 (SEQ ID NO:3), CEP290 (SEQ ID NO:4), KRT4 (SEQ ID NO:5), LOC126076011 (SEQ ID NO:6), NCKAP5 (SEQ ID NO:7), LAMB4 (SEQ ID NO:8), NPC1L1 (SEQ ID NO:9), ADGRD2 (SEQ ID NO:10), NINJ1 (SEQ ID NO:11), AHNAK2 (SEQ ID NO:12), CATSPERB (SEQ ID NO:13). PCNXL4 (SEQ ID NO:14), SYNE2 (SEQ ID NO:15), NLRP12 (SEQ ID NO:16), LOC126086768 (SEQ ID NO:17), WDR90 (SEQ ID NO:18), BRCA2 (SEQ ID NO:19), PRKDC (SEQ ID NO:20), VPS13B (SEQ ID NO:21), PSAP (SEQ ID NO:22), SEC31B (SEQ ID NO:23), KRT28 (SEQ ID NO:24), KRT35 (SEQ ID NO:25), KRT40 (SEQ ID NO:26), MYH4 (SEQ ID NO:27), PIRT (SEQ ID NO:28), PKD1L2 (SEQ ID NO:29), RP1L1 (SEQ ID NO:30), CXorf58 (SEQ ID NO:31), LOC126068772 (SEQ ID NO:32), LOC126069872 (SEQ ID NO:33), THRAP3 (SEQ ID NO:34), CENPC1 (SEQ ID NO:35), DSPP (SEQ ID NO:36), FGF5 (SEQ ID NO:37), CATSPERG (SEQ ID NO:38), MYH1 (SEQ ID NO:39), MYH13 (SEQ ID NO:40), ATRIP (SEQ ID NO:41), and TGM3 (SEQ ID NO:42).

Embodiment 24 is the transgenic animal of any one of embodiments 21 to 23, wherein the transgenic animal comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 woolly mammoth (Mammuthus primigenius) gene variants.

Embodiment 25 is the transgenic animal of any one of embodiments 21 to 24, wherein the transgenic animal further comprises a deletion of at least one nucleotide sequence upstream of a transcription start site of LOC126071805, WASH complex subunit 4 (WASHC4), LOC126075532, LOC126075533, alpha-fetoprotein (AFP), LOC126079103, LOC126079327, LOC126079327, LOC126080333, LOC126080367, LOC126080369. LOC126080369, LOC126080733, distal-less homeobox 6 (DLX6), extracellular matrix protein 2 (ECM2), LOC126085059, LOC126085481, LOC126086151, LOC126086152, LOC126086293, LOC126058227, LOC126058390, LOC126058396, forkhead box H1 (FOXH1), LOC126060018, LOC126060570, coiled-coil domain containing 15 (CCDC15), INO80 complex subunit B (INO80B), LOC126062579, LOC126063153, LOC126063990, LOC126063991, LOC126066513, LOC126066877, acyl-CoA wax alcohol acyltransferase 1 (AWAT1), transmembrane protein 187 (TMEM187), and LOC126069912, wherein the deleted nucleic acid sequence upstream of:
(a) LOC126071805 comprises SEQ ID NO:43,
(b) WASHC4 comprises SEQ ID NO:44,
(c) LOC126075532 comprises SEQ ID NO:45,
(d) LOC126075533 comprises SEQ ID NO:46,
(e) AFP comprises SEQ ID NO:47,
(f) LOC126079103 comprises SEQ ID NO:48,
(g) LOC126079327 comprises SEQ ID NO:49,
(h) LOC126079327 comprises SEQ ID NO:50,
(i) LOC126080333 comprises SEQ ID NO:51,
(j) LOC126080367 comprises SEQ ID NO:52,
(k) LOC126080369 comprises SEQ ID NO:53,
(l) LOC126080369 comprises SEQ ID NO:54,
(m) LOC126080733 comprises SEQ ID NO:55,
(n) DLX6 comprises SEQ ID NO:56,
(o) ECM2 comprises SEQ ID NO:57,
(p) LOC126085059 comprises SEQ ID NO:58,
(q) LOC126085481 comprises SEQ ID NO:59,
(r) LOC126086151 comprises SEQ ID NO:60,
(s) LOC126086152 comprises SEQ ID NO:61,
(t) LOC126086293 comprises SEQ ID NO:62,
(u) LOC126058227 comprises SEQ ID NO:63,
(v) LOC126058390 comprises SEQ ID NO:64,
(w) LOC126058396 comprises SEQ ID NO:65,
(x) FOXH1 comprises SEQ ID NO:66,
(y) LOC126060018 comprises SEQ ID NO:67,
(z) LOC126060570 comprises SEQ ID NO:68,
(aa) CCDC15 comprises SEQ ID NO:69,
(bb) INO80B comprises SEQ ID NO:70,
(cc) LOC126062579 comprises SEQ ID NO:71,
(dd) LOC126063153 comprises SEQ ID NO: 72,
(ee) LOC126063990 comprises SEQ ID NO:73,
(ff) LOC126063991 comprises SEQ ID NO:74,
(gg) LOC126066513 comprises SEQ ID NO: 75,
(hh) LOC126066877 comprises SEQ ID NO:76,
(ii) AWAT1 comprises SEQ ID NO:77,
(jj) TMEM187 comprises SEQ ID NO: 78, and
(kk) LOC126069912 comprises SEQ ID NO:79.

Embodiment 26 is the transgenic animal of any one of embodiments 21 to 25, wherein the transgenic animal fails to express an endogenous homologue of at least one of GPR98, MACF1, ADARB2. CEP290, KRT4, LOC126076011, NCKAP5, LAMB4, NPC1L1, ADGRD2, NINJ1, AHNAK2, CATSPERB. PCNXL4, SYNE2, NLRP12, LOC126086768, WDR90. BRCA2, PRKDC, VPS13B, PSAP, SEC31B, KRT28, KRT35, KRT40, MYH4, PIRT, PKD1L2, RP1L1, CXorf58, LOC126068772, LOC126069872, THRAP3, CENPC1, DSPP, FGF5, CATSPERG, MYH1, MYH13, ATRIP, and TGM3.

Embodiment 27 is the transgenic animal of embodiment 26, wherein the transgenic animal fails to express the endogenous homologue of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 of GPR98, MACF1, ADARB2, CEP290, KRT4, LOC126076011, NCKAP5, LAMB4, NPC1L1, ADGRD2, NINJ1, AHNAK2, CATSPERB, PCNXL4, SYNE2, NLRP12, LOC126086768, WDR90, BRCA2, PRKDC, VPS13B, PSAP, SEC31B, KRT28, KRT35, KRT40, MYH4, PIRT, PKDIL2, RP1L1, CXorf58, LOC126068772, LOC126069872, THRAP3, CENPC1, DSPP, FGF5, CATSPERG, MYH1, MYH13, ATRIP, and TGM3.

Embodiment 28 is the transgenic animal of any one of embodiments 21 to 27, wherein the transgenic animal is an elephant.

Embodiment 29 is the transgenic animal of embodiment 28, wherein the elephant is selected from an Asian elephant (Elephas maximus), an African elephant (Loxodonta africana), an African forest elephant (Loxodonta cyclotis), and a Bornean elephant (Elephas maximus borneensis).

### EXAMPLES

### Example 1: Identification of Woolly Mammoth Variants

Variants in 46 woolly mammoths, 12 Asian elephants and 27 African elephants were identified using the mEleMax1 reference genome. Reads are trimmed using AdapterRemoval¹ or fastp, to trim low quality (qual <25) ends of reads and to remove reads <35 bp. Contaminants were detected using Kraken with confidence of 0.8, using the (minikraken2_v2_8 GB 201904) Kraken precompiled database. Unclassified trimmed reads were aligned to a reference genome using BWA Aln (with seed of 16,500, maximum edit distance of 0.01 and maximum gap opens of 2), Duplicate reads were optionally marked using PaleoMIX² (Mammoth samples) and Picard MarkDuplicates (Elephant samples). Binary alignment maps (BAMs) from the same sample generated by multiple runs were merged using samtools³. Alignment quality was assessed using QualiMap⁴, DamageProfiler⁵, and MultiQC⁶. Germline variants were detected using the following tools Samtools/Bcftools³ and GATK4⁷. Genotyping of GVCF files was determined using GLnexus⁸. Variant effects were determined using SNPEff⁹. Variants were filtered if they were also observed in Asian and African elephant samples and across Asian elephant repeat and CpG island regions.

Mammoth fixed variants were identified, and it was determined that the mammoth fixed variants must (i) be homozygous in all woolly mammoths with a called genotype with read depth >5; (ii) not observed in Asian and African elephants; (iii) not in repeat or CpG island regions.

An mEleMax1 siftDB was created. Missense variants were annotated with the siftDB to predict effect. Genes were ranked by their sum of Sift scores for missense variants found in at least 5 mammoths.

A pairwise whole genome alignment was generated between the Asian elephant reference genome, mEleMax1, and the human reference genome, Hg38 from UCSC using SegAlign.¹⁰ With this pairwise alignment, CrossMap Region was used to convert the genome coordinates of several publicly available datasets.¹¹ From UCSC, TF Clusters, an ENCODE 3 regulatory track in Hg38 (Transcription Factor ChIP-Seq Clusters in 340 TFs in 129 cell types) was downloaded.¹²⁻¹⁴ From UCSC, data from NCBI RefSeq Functional Elements and ENCODE cCREs (candidate cis-regulatory elements) in Hg38 and Mm10 were also downloaded.^{14,15} These steps were repeated for the mouse genome, Mm10. The result was a dataset of putative regulatory elements in the Asian Elephant genome derived from experimental data in humans and mice. BEDTools were then used to intersect to identify the putative regulatory regions that overlapped with fixed Mammoth variants.¹⁶ Next, the most proximal mEleMax1 gene to each putative regulatory region was identified with BEDTools closest.¹⁶ These regulatory regions were then filtered for proximity within 2 kb of the nearest gene.

From the data analysis, a list of protein coding gene variants were identified in woolly mammoth genomes that were not present in the comparative elephant genome. The protein coding woolly mammoth gene variants are provided in Table 1.

TABLE 1
Protein Coding Gene Variants

| | Chrom. | | | Starting | Ending | SEQ |
|---|---|---|---|---|---|---|
| Gene Symbol | number | Chrom. ID | Modification | Coordinate | Coordinate | ID NO: |
| GPR98 | 2 | NC_064820.1 | Modified CDS | 97230878 | 97908100 | 1 |
| MACF1 | 3 | NC_064821.1 | Modified CDS | 80737842 | 81115459 | 2 |
| ADARB2 | 4 | NC_064822.1 | Modified CDS | 18022109 | 18355792 | 3 |
| CEP290 | 4 | NC_064822.1 | Modified CDS | 151681894 | 151780870 | 4 |
| KRT4 | 4 | NC_064822.1 | Modified CDS | 108325090 | 108331363 | 5 |
| LOC126076011 | 4 | NC_064822.1 | Modified CDS | 180667685 | 180691566 | 6 |
| NCKAP5 | 6 | NC_064824.1 | Modified CDS | 27394062 | 28416452 | 7 |
| LAMB4 | 8 | NC_064826.1 | Knockout | 41478000 | 41585567 | 8 |
| NPC1L1 | 8 | NC_064826.1 | Modified CDS | 120589354 | 120607506 | 9 |
| ADGRD2 | 9 | NC_064827.1 | Knockout | 75487744 | 75508679 | 10 |
| NINJ1 | 9 | NC_064827.1 | Modified CDS | 111444027 | 111457619 | 11 |
| AHNAK2 | 10 | NC_064828.1 | Modified CDS | 119224622 | 119259457 | 12 |
| CATSPERB | 10 | NC_064828.1 | Modified CDS | 105215890 | 105351340 | 13 |
| PCNXL4 | 10 | NC_064828.1 | Modified CDS | 72497095 | 72522071 | 14 |
| SYNE2 | 10 | NC_064828.1 | Modified CDS | 76215041 | 76519751 | 15 |
| NLRP12 | 11 | NC_064829.1 | Knockout | 93279727 | 93312039 | 16 |
| LOC126086768 | 12 | NC_064830.1 | Knockout | 54283985 | 54284917 | 17 |
| WDR90 | 12 | NC_064830.1 | Knockout | 57869745 | 57884421 | 18 |
| BRCA2 | 14 | NC_064832.1 | Modified CDS | 95272431 | 95336586 | 19 |
| PRKDC | 15 | NC_064833.1 | Modified CDS | 88372166 | 88693020 | 20 |
| VPS13B | 15 | NC_064833.1 | Modified CDS | 42126454 | 43039849 | 21 |
| PSAP | 16 | NC_064834.1 | Modified CDS | 15692853 | 15734238 | 22 |
| SEC31B | 16 | NC_064834.1 | Knockout | 52143767 | 52170359 | 23 |
| KRT28 | 19 | NC_064837.1 | Modified CDS | 47884097 | 47894712 | 24 |
| KRT35 | 19 | NC_064837.1 | Modified CDS | 48552497 | 48556287 | 25 |
| KRT40 | 19 | NC_064837.1 | Modified CDS | 48071439 | 48076172 | 26 |
| MYH4 | 19 | NC_064837.1 | Modified CDS | 10054936 | 10082689 | 27 |
| PIRT | 19 | NC_064837.1 | Modified CDS | 9612650 | 9613063 | 28 |
| PKD1L2 | 21 | NC_064839.1 | Knockout | 47029360 | 47127389 | 29 |
| RP1L1 | 22 | NC_064840.1 | Modified CDS | 41260152 | 41284034 | 30 |
| CXorf58 | X | NC_064846.1 | Modified CDS | 155215297 | 155245281 | 31 |
| LOC126068772 | X | NC_064846.1 | Knockout | 110971028 | 110976128 | 32 |
| LOC126069872 | Y | NC_064847.1 | Knockout | 6371001 | 6440341 | 33 |
| THRAP3 | 3 | NC_064821.1 | Modified CDS | 77643843 | 77665544 | 34 |
| CENPC1 | 5 | NC_064823.1 | Modified CDS | 91999666 | 92104092 | 35 |
| DSPP | 5 | NC_064823.1 | Modified CDS | 67791228 | 67795372 | 36 |
| FGF5 | 5 | NC_064823.1 | Knockout | 76557733 | 76582982 | 37 |
| CATSPERG | 11 | NC_064829.1 | Modified CDS | 113209697 | 113244241 | 38 |
| MYH1 | 19 | NC_064837.1 | Modified CDS | 9997859 | 10021861 | 39 |
| MYH13 | 19 | NC_064837.1 | Modified CDS | 10190826 | 10268473 | 40 |
| ATRIP | 20 | NC_064838.1 | Modified CDS | 52991911 | 53007157 | 41 |
| TGM3 | 25 | NC_064843.1 | Modified CDS | 40795303 | 40839811 | 42 |

Additionally, a collection of regulatory element variants, i.e., deletions, were identified in the woolly mammoth genomes that were not present in the comparative elephant genome. The regulatory target variants are provided in Table 2.

TABLE 2
Regulatory Element Variants

| | Chrom. | | | Starting | Ending | SEQ ID |
|---|---|---|---|---|---|---|
| Gene Symbol | number | Chrom. ID | Modification | Coordinate | Coordinate | NO: |
| LOC126071805 | 3 | NC_064821.1 | Deletion | 411676 | 412745 | 43 |
| WASHC4 | 4 | NC_064822.1 | Deletion | 171996688 | 171997927 | 44 |
| LOC126075532 | 4 | NC_064822.1 | Delction | 110712383 | 110715064 | 45 |
| LOC126075533 | 4 | NC_064822.1 | Deletion | 110732816 | 110736147 | 46 |
| AFP | 5 | NC_064823.1 | Deletion | 84233436 | 84233976 | 47 |
| LOC126079103 | 7 | NC_064825.1 | Deletion | 55433944 | 55437172 | 48 |
| LOC126079327 | 7 | NC_064825.1 | Deletion | 55693317 | 55696232 | 49 |
| LOC126079327 | 7 | NC_064825.1 | Deletion | 55699142 | 55699867 | 50 |
| LOC126080333 | 7 | NC_064825.1 | Deletion | 110255368 | 110256024 | 51 |
| LOC126080367 | 7 | NC_064825.1 | Deletion | 111451618 | 111452860 | 52 |
| LOC126080369 | 7 | NC_064825.1 | Deletion | 111466259 | 111466931 | 53 |
| LOC126080369 | 7 | NC_064825.1 | Deletion | 111470639 | 111475865 | 54 |
| LOC126080733 | 7 | NC_064825.1 | Deletion | 113462642 | 113463310 | 55 |
| DLX6 | 8 | NC_064826.1 | Deletion | 67097379 | 67097982 | 56 |
| ECM2 | 9 | NC_064827.1 | Deletion | 110846893 | 110848763 | 57 |
| LOC126085059 | 11 | NC_064829.1 | Deletion | 89028871 | 89029532 | 58 |
| LOC126085481 | 11 | NC_064829.1 | Deletion | 37543079 | 37544258 | 59 |
| LOC126086151 | 11 | NC_064829.1 | Deletion | 87844515 | 87845207 | 60 |
| LOC126086152 | 11 | NC_064829.1 | Deletion | 87894678 | 87895267 | 61 |
| LOC126086293 | 11 | NC_064829.1 | Deletion | 107934503 | 107935958 | 62 |
| LOC126058227 | 14 | NC_064832.1 | Deletion | 2404741 | 2405638 | 63 |
| LOC126058390 | 14 | NC_064832.1 | Deletion | 2362484 | 2362989 | 64 |
| LOC126058396 | 14 | NC_064832.1 | Deletion | 2525557 | 2527369 | 65 |
| FOXH1 | 15 | NC_064833.1 | Deletion | 490002 | 490510 | 66 |
| LOC126060018 | 16 | NC_064834.1 | Deletion | 85207224 | 85207842 | 67 |
| LOC126060570 | 17 | NC_064835.1 | Deletion | 25448607 | 25449799 | 68 |
| CCDC15 | 17 | NC_064835.1 | Deletion | 27205355 | 27207828 | 69 |
| INO80B | 17 | NC_064835.1 | Deletion | 61371908 | 61372663 | 70 |
| LOC126062579 | 19 | NC_064837.1 | Deletion | 388998 | 390604 | 71 |
| LOC126063153 | 19 | NC_064837.1 | Deletion | 39536524 | 39537102 | 72 |
| LOC126063990 | 20 | NC_064838.1 | Deletion | 79145135 | 79146220 | 73 |
| LOC126063991 | 20 | NC_064838.1 | Deletion | 79130190 | 79130940 | 74 |
| LOC126066513 | 23 | NC_064841.1 | Deletion | 18025548 | 18026085 | 75 |
| LOC126066877 | 24 | NC_064842.1 | Deletion | 65054280 | 65055172 | 76 |
| AWAT1 | X | NC_064846.1 | Deletion | 99815327 | 99817444 | 77 |
| TMEM187 | X | NC_064846.1 | Deletion | 2350623 | 2351165 | 78 |
| LOC126069912 | Unplaced | NW_026060255.1 | Deletion | 2318 | 4646 | 79 |

### Example 2: Targeted Editing of FGF5

FGF5 encodes a protein within the FGF family, which is involved in embryonic development, cell growth, and tissue repair among other biological functions. This gene has been closely associated with an inhibitory action on hair elongation via promotion of movement from anagen to catagen phase in hair follicle cycle.

FGF5 has been closely associated with hair development, particularly in the anagen-catagen hair follicle cycle junction. Mutations resulting in a truncated variant of this protein due to a single nucleotide deletion and subsequent premature stop codon formation have been associated with a phenotypic presentation of uncharacteristically long hair for multiple species. This primarily appears to affect only certain hair types (i.e., down in rabbits and facial hair in humans). This truncated protein has not been specifically linked to detrimental developmental phenotypes.

CRISPR (such as, e.g., CRISPR-Cas9 or CRISPR-Cas12) may be used to knock-out FGF5 in the cell to be edited. The CRISPR (such as, e.g., CRISPR-Cas9 or CRISPR-Cas12) system may include a targeting guide with the following sequence TGAGGAAAGAAGCAGGAGGG (SEQ ID NO:80). An Asian elephant (Elephas maximus) cell is edited to incorporate an indel in the FGF5 gene.

The disruption of FGF5 is involved in a variety of biological processes, including embryonic development, cell growth, morphogenesis, tissue repair, tumor growth and invasion. This gene was identified as an oncogene, which confers transforming potential when transfected into mammalian cells. Targeted disruption of the homolog of this gene in mouse resulted in the phenotype of abnormally long hair, which suggests a function as an inhibitor of hair elongation. Alternatively spliced transcript variants encoding different isoforms have been identified. Gene editing via methods, such as, e.g., a CRISPR (such as, e.g., CRISPR-Cas9 or CRISPR-Cas12) based knock-out mechanism at a SNP site observed in mammoth to elephant genome comparisons is anticipated to lead to a truncated variant of FGF5. The phenotypic response to this disrupted gene is anticipated to be an organism with substantially elongated hair due to the reduced system capacity to promote movement from anagen to catagen phase in hair follicles.

### Example 3: Replacing the NINJ1 and NCKAP5 Genes and Testing the Integration Efficiency of attB Sites Via RNPs and ssODNs

### Transfection Reagents

The following reagents were thawed on ice as needed: (a) Editor(s) Alt-R™ S.p. Cas9 nuclease V3 protein (1 mg/ml), Alt-R™ A.s. Cas12a (1 mg/ml), or plasmid editors: (b) gRNA(s): 100 μM sgRNA (Synthego; Redwood City, CA) dissolved in 15 μl nuclease free H2O; (c) Donor(s): Homology-directed repair (HDR) donor plasmid or linear dsDNA (PCR purified): (d) Alt-R™ HDR-Enhancer V2: (e) Pifithrin-α (P53 inhibitor). The RNP buffers and transfection cuvettes were located and include the following: (a) P3 primary cell Nucleofector™ solution (LONZA™ P3 Primary Cell 4D-NUCLEOFECTOR™ X Kit L (Lonza kit; Lonza; Basel, Switzerland)); (b) Supplement 1 buffer (Lonza); and (c) 100 μL Nucleocuvette™ (Lonza).

### Prepared Transfection Reagents

The hybridization of editor protein with sgRNA to form Cas9-ribonucleoproteins (RNPs) was performed by combining equal volumes Alt-R™ S.p. Cas9 nuclease V3 protein (1 μg/μl), 100 μM sgRNA, and nuclease-free water for at least 10 minutes. Each sgRNA or crRNA was hybridized independently as the editor protein has different affinities for different sgRNA sequences.

A standard volume of 2.5 μl of each reagent was used, except for NCKAP5 where 3.33 μl and 1.67 μl was used for each RNP to compensate for differences in efficiency between RNPs. The P3/S1 buffer was prepared (82% P3, 18% S1). This buffer was used to resuspend cells directly prior to transfection. 2.5 μg of midi-prepped HDR donor plasmid was used. The P3/S1 buffer was prepared (82% P3, 18% S1). This buffer was used to resuspend cells directly prior to transfection. 100 μl of P3/S1 buffer was used for a single reaction.

### Plasmid-Based Editor Reactions

The equation for calculating plasmid volume added to the editor reactions is as follows: plasmid volume (μl)=desired plasmid amount (ng)/plasmid concentration (ng/μl). The plasmid-based editing reaction reagents did not require incubation and donors were mixed with editors and gRNAs prior to adding to cells.

### Transfection Reaction

Cells were healthy, growing, and near confluency prior to the transfection. At least 500,000 cells, ideally 1,000,000 cells or more, were used for each transfection reaction. To enrich for cells with the replacement, FACS was used.

To prepare the cells for transfection, media was aspirated from the cells using a 2 ml serological pipette connected to vacuum pressure and a sterile nonfiltered tip. The cells were washed in 1×PBS rocking back and forth for full coverage of the cells. The 1×PBS was then aspirated from the cells, and then the cells were washed again in 1×PBS. 0.25% trypsin was added directly to the cells and the culture dish was rocked back and forth for full coverage. The cells were incubated for 2-3 minutes in trypsin while rocking at 37° C., 5% CO₂ and were checked for full release using a microscope. An equal volume of 1:1 media added to cells to neutralize the trypsin and the cells were aliquoted to a tube for centrifugation. The cells were centrifuged at 300×g for 10 minutes. A T25 flask was prepared by adding 5 ml of 1:1 media, 1 μl of pifithrin-α, and 7.14 μl of Alt-R™ HDR-Enhancer V2. The supernatant was removed from the centrifuged cells, and the cells were resuspended with 1 ml of 1:1 media. 10 μl of the resuspended cells were added to 10 d of trypan blue for each sample, and the cells were counted by adding 10 d to the cell counter. The remaining cells were centrifuged at 300×g for 10 minutes, the supernatant was removed, and cells were resuspended in P3/S1 buffer. The guide/editor/H₂O master mix and HDR donor were added to the P3/S1 suspended cells to a total volume of 100 μl. The 100 μl was added to a 100 μl large cuvette, and the cells were transfected using the Lonza nucleofector. The transfected cells were transferred to a T25 flask and an additional 100 μl of 1:1 media was added from the flask into the cuvette and the remaining cells were transferred to the flask. The flasks were incubated for 24 hours, and media was changed using 1:1 media only.

### 1:1 Standard Growth Media Preparation

The following were combined and filter sterilized for 1 liter of “1:1 media”: 300 ml of Ham's F12 (Thermo Fisher Scientific, Waltham, MA), 200 ml of HI FBS (Thermo Fisher), 5 ml of 2 mM GlutaMAX (Thermo Fisher), 5 ml of 100×NEAA (Thermo Fisher), 5 ml of 100× Anti/Anti (Thermo Fisher), 50 pd of 100 ng/μl bFGF (StemCell Technologies, Vancouver, BC, Canada), 50 μl of 100 μg/μl EGF (StemCell Technologies), 1 ml of 55 mM BME (Thermo Fisher), 5 μl of 50 mg/ml ascorbic acid (Sigma-Aldrich, Burlington, MA), 500 ml of EGM-2 Bullet kit media (Lonza, Walkersville, MD), and EGM-2 Bullet kit supplements (Lonza).

### Populations of Cells Enriched by FACS

FACS was performed to enrich the cell population for stable fluorescent protein (e.g., GFP) expression. Transient expression was typically done by 7 days post transfection and fluorescent protein expression greater than 8 days post transfection typically represents stable integrants. For FACS enrichment, the plates were set-up for receiving sorted cell samples by adding a 1:1 mixture of 1:1 media and filtered FBS media. Cell pellets were generated as described above. The samples were stored on ice and transported directly to FACS facility along with tissue culture plate(s). The cells were sorted into the plates. The sorted plates were transported directly back to the incubator for culturing. The cells were examined on the following day to confirm population adherence and expansion. The pass rate and filters used were recorded. For 96 well and 384 well plates, there was no need to change media for the first 5 days post transfection. On day 5, and every 2-3 days after until the cells were passaged or the well was full, 1:1 media was directly added to the wells without replacing media to give the cells more time in FBS rich media before weaning the cells off of the media.

### Sequence Verification

Following the steps provided above for pelleting cells, less than 1,000,000 cells and greater than 20,000 cells were pelleted for optimal cell lysis. The supernatant was removed and the cells were frozen at −20° C. to continue to lysis/DNA extraction.

For lysis and DNA extraction, lysis stock buffer was made by combining protein degrader (Life Sciences, Gene Art Genomic Cleavage Kit) and Quick Extract DNA Extraction solution in the ratio of 1 μl/25 μl, respectively. Lysis buffer was added to the tube containing the dry and thawed cell pellet. The cell pellet was vortexed for 10-15 seconds. The contents were transferred to a tube and placed on a 65° C. heat block or thermal cycler for 6 minutes. The tube was vortexed for 10-15 seconds, and then transferred to a 98° C. heat block for 2 minutes. The samples were lysed and used in subsequent PCR reactions using 5 pd or less of lysate.

Genotyping Replacements with PCR

Primers for the first round of PCR were designed with one primer outside the homology arms and the second primer binding to a genomic/donor locus prior to the location of the reporter insertion. Primers for nested PCR of each end of the replacement were designed so that with a second round of PCR all the variants and cut sites can be used in a PCR reaction and Sanger sequenced. The Q5 polymerase was recommended to improve sequence quality and for improved long range PCR amplification. DNA that was a high concentration and high quality was preferred. For the PCR1 reaction mix, 20 d of a reaction mixture containing the following was mixed: (a) 10 μl Q5CR High-Fidelity DNA polymerase (2×); 0.2 μl forward and 0.2 μl reverse primer (10 μM); (c) 1 μl of each cell lysate; (d) filled to 20 μl with nuclease free water. For each unique edited amplicon, a positive control was prepared. A negative control without cell lysate was prepared as well. For the PCR1 reaction, the following conditions were used in the thermal cycler: 105° C. cover temperature; 20 μl volume: 1×98° C. for 2 minutes; 20×: 98° C. for 10 seconds, primer specific annealing temperature for 30 seconds, 72° C. for about 30 seconds per kilobase of DNA: 1×72° C. for 30 seconds per kilobase: 4° C. infinite hold. The PCR product was visualized on a gel.

For the PCR 2 reaction mix, a 30 μl reaction mixture containing the following was mixed: (a) 15 μl Q5® High-Fidelity DNA polymerase (2×); 0.3 μl forward and 0.3 μl reverse primer (10 μM); (c) 1 μl of round 1 PCR product; (d) filled to 30 μl with nuclease free water. For each unique edited amplicon, a positive control was prepared. A no-template negative control was also prepared that had a round 1 PCR product with no DNA added. For the PCR2 reaction, the following conditions were used in the thermal cycler: 105° C. for the cover for 30 μl volume; 1×98° C. for 2 minutes; 20×: 98° C. for 10 seconds, primer specific annealing temperature for 15 seconds, and 72° C. for about 30 seconds per kilobase of DNA; 1×4° C. for infinite; save PCR product for Sanger sequencing.

Gel verification was performed for the PCR products from round 1 and 10×PCR reactions. Samples were submitted for Sanger sequencing. Any samples that failed to have a CRL over 50 or a QS above 30 were noted. The sequences were aligned to a target sequence and the files were visually inspected to make sure that they align with where they were expected to and to check for other potential issues in quality or coverage. Synthego ICE analysis was performed according to the template and instructions available online.

### Clonal Isolation and Cryobanking

Based on the sequence verification, it was determined if further enrichment was required by verifying that the desired edit was present and the percentage of population with that edit. For samples that were verified to have desired edit that did not need further enrichment, steps for clonal isolation and enriched population and genotyping as described above were repeated. For samples that were verified to have the desired edit and did not need further enrichment, the samples were expanded and cryobanked as modified cell lines.

Edit Checkpoint. Clonal Outgrowth, and WGS-Based on/Off Targeting

The modified cell line was expanded and utilized for repeated application. Once greater than 5 verified edits were performed, at least 1×10⁶ cells were allotted for WGS analysis to determine on/off targeting. For preparation of WGS samples, the cells were spun at 300×g and the supernatant was removed. Then the cell pellet was flash froze with liquid nitrogen and stored under liquid nitrogen conditions.

### CONCLUSION

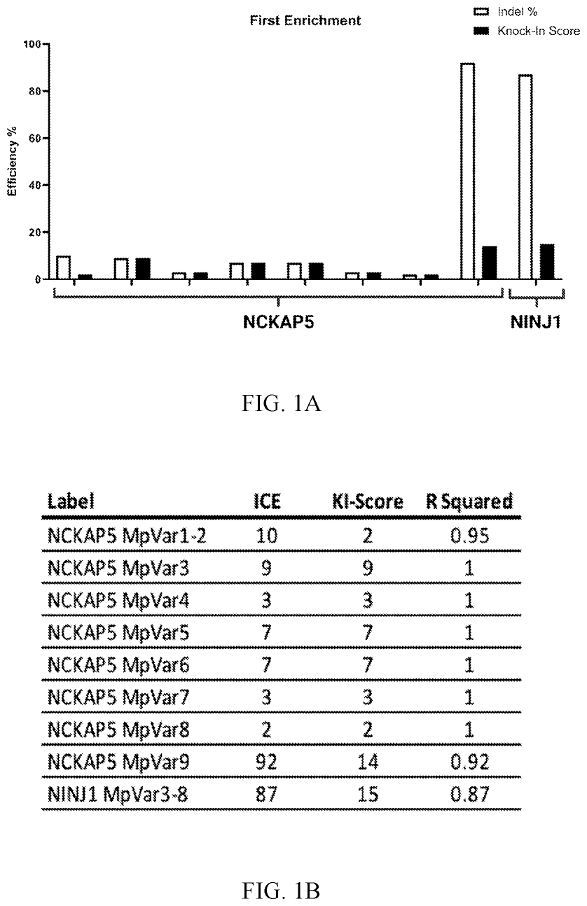

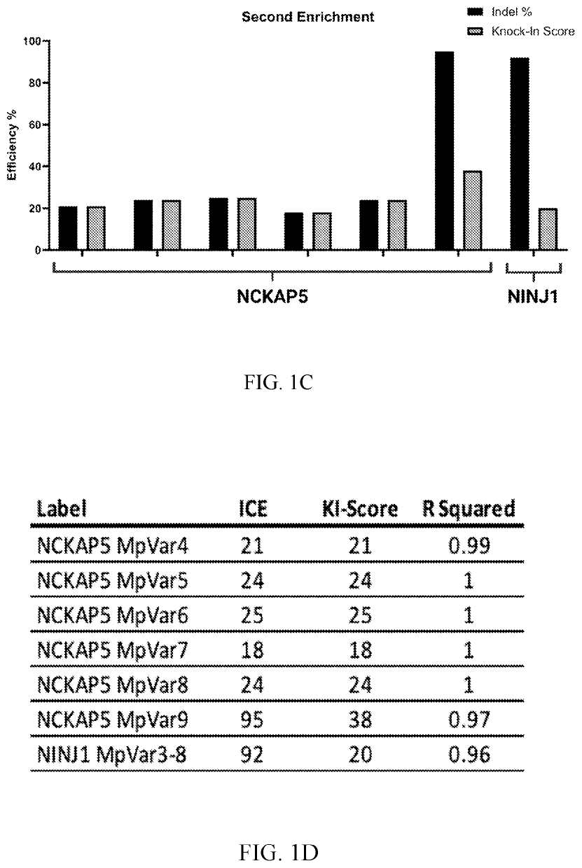

Gene replacement of the NINJ1 and NCKAP5 genes was performed sequentially in the same cell line and the indel and knock-in scores for the first and second enrichments of these cell lines are shown in FIGS. 1A-1D. This method was successful in generating a cell line that has approximately 20% replacement for all intended mammoth specific substitutions across the NINJ1 and NCKAP5 genes based on genotyping of parental lines.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

### REFERENCES

1. Schubert, M., Lindgreen, S. & Orlando, L. AdapterRemoval v2: rapid adapter trimming, identification, and read merging. BMC Res. Notes 9, 88 (2016).
2. Schubert. M. et al. Characterization of ancient and modern genomes by SNP detection and phylogenomic and metagenomic analysis using PALEOMIX. Nat. Protoc. 9, 1056-1082 (2014).
3. Li, H. et al. The Sequence Alignment/Map format and SAMtools. Bioinformatics 25, 2078-2079 (2009).
4. García-Alcalde, F. et al. Qualimap: evaluating next-generation sequencing alignment data. Bioinformatics 28, 2678-2679 (2012).
5. Jónsson, H., Ginolhac, A., Schubert, M., Johnson, P. L. F. & Orlando, L. mapDamage2.0: fast approximate Bayesian estimates of ancient DNA damage parameters. Bioinformatics 29, 1682-1684 (2013).
6. Ewels, P., Magnusson, M., Lundin, S. & Käller, M. MultiQC: summarize analysis results for multiple tools and samples in a single report. Bioinformatics 32, 3047-3048 (2016).
7. McKenna, A. et al. The Genome Analysis Toolkit: a MapReduce framework for analyzing next-generation DNA sequencing data. Genome Res. 20, 1297-1303 (2010).
8. Lin, M. F. et al. GLnexus: joint variant calling for large cohort sequencing. http://biorxiv.org/lookup/doi/10.1101/343970 (2018) doi:10.1101/343970.
9. Cingolani, P. et al. A program for annotating and predicting the effects of single nucleotide polymorphisms, SnpEff Fly (Austin) 6, 80-92 (2012).
10. Goenka, S. D., Turakhia, Y., Paten, B. & Horowitz, M. SegAlign: A Scalable GPU-Based Whole Genome Aligner. in SC20: International Conference for High Performance Computing, Networking, Storage and Analysis 1-13 (IEEE, 2020). doi: 10.1109/SC41405.2020.00043.
11. Zhao, H. et al. CrossMap: a versatile tool for coordinate conversion between genome assemblies. Bioinformatics 30, 1006-1007 (2014).
12. ENCODE Project Consortium. An integrated encyclopedia of DNA elements in the human genome. Nature 489, 57-74 (2012).
13. Luo, Y. et al. New developments on the Encyclopedia of DNA Elements (ENCODE) data portal. Nucleic Acids Res. 48, D882-D889 (2020).
14. The ENCODE Project Consortium et al. Expanded encyclopaedias of DNA elements in the human and mouse genomes. Nature 583, 699-710 (2020).
15. Pruitt, K. D. et al. RefSeq: an update on mammalian reference sequences. Nucleic Acids Res. 42, D756-763 (2014).
16. Quinlan, A. R. & Hall. I. M. BEDTools: a flexible suite of utilities for comparing genomic features. Bioinformatics 26, 841-842 (2010).

1. An isolated nucleic acid sequence comprising a nucleotide sequence having at least 80%, at least 85%, at least 90%, at least 95%, at least 96%, at least 97%, at least 98%, or at least 99% identity to at least one of G protein-coupled receptor 98 (GPR98) (SEQ ID NO:1), microtubule actin crosslinking factor 1 (MACF1) (SEQ ID NO:2), adenosine deaminase RNA specific B2 (ADARB2) (SEQ ID NO:3), centrosomal protein 290 (CEP290) (SEQ ID NO:4), keratin 4 (KRT4) (SEQ ID NO:5), LOC126076011 (SEQ ID NO:6), NCK associated protein 5 (NCKAP5) (SEQ ID NO:7), laminin subunit beta 4 (LAMB4) (SEQ ID NO:8), Niemann-Pick C1-like 1 (NPC1L1) (SEQ ID NO:9), adhesion G protein-coupled receptor D2 (ADGRD2) (SEQ ID NO:10), ninjurin 1 (NINJ1) (SEQ ID NO:11), AHNAK nucleoprotein 2 (AHNAK2) (SEQ ID NO:12), cation channel sperm associated auxiliary subunit beta (CATSPERB) (SEQ ID NO:13), pecanex-like 4 (PCNXL4) (SEQ ID NO:14), spectrin repeat containing nuclear envelope protein 2 (SYNE2) (SEQ ID NO:15), NLR family pyrin domain containing 12 (NLRP12) (SEQ ID NO:16), LOC126086768 (SEQ ID NO:17), WD repeat domain 90 (WDR90) (SEQ ID NO:18), breast cancer 2 (BRCA2) (SEQ ID NO:19), protein kinase, DNA-activated catalytic subunit (PRKDC) (SEQ ID NO:20), vacuolar protein sorting 13 homolog B (VPS13B) (SEQ ID NO:21), prosaposin (PSAP) (SEQ ID NO:22), SEC31 homolog B (SEC31B) (SEQ ID NO:23), keratin 28 (KRT28) (SEQ ID NO:24), keratin 35 (KRT35) (SEQ ID NO:25), keratin 40 (KRT40) (SEQ ID NO:26), myosin heavy chain 4 (MYH4) (SEQ ID NO:27), phosphoinositide interacting regulator of TRP (PIRT) (SEQ ID NO:28), polycystin 1 like 2 (PKD1L2) (SEQ ID NO:29), retinitis pigmentosa 1-like protein (RP1L1) (SEQ ID NO:30), chromosome X open reading frame 58 (CXorf58) (SEQ ID NO:31), LOC126068772 (SEQ ID NO:32), LOC126069872 (SEQ ID NO:33), thyroid hormone receptor associated protein 3 (THRAP3) (SEQ ID NO:34), centromere protein C 1 (CENPC1) (SEQ ID NO:35), dentinogenesis and dentin sialophosphoprotein (DSPP) (SEQ ID NO:36), fibroblast growth factor 5 (FGF5) (SEQ ID NO:37), cation channel sperm associated auxiliary subunit gamma (CATSPERG) (SEQ ID NO:38), myosin heavy chain 1 (MYH1) (SEQ ID NO:39), myosin heavy chain 13 (MYH13) (SEQ ID NO:40), ATR interacting protein (ATRIP) (SEQ ID NO:41), and transglutaminase 3 (TGM3) (SEQ ID NO:42).

2. The isolated nucleic acid sequence of claim 1, wherein the nucleotide sequence comprises at least one of GPR98 (SEQ ID NO:1), MACF1 (SEQ ID NO:2), ADARB2 (SEQ ID NO:3), CEP290 (SEQ ID NO:4), KRT4 (SEQ ID NO:5), LOC126076011 (SEQ ID NO:6), NCKAP5 (SEQ ID NO:7), LAMB4 (SEQ ID NO:8), NPC1L1 (SEQ ID NO:9), ADGRD2 (SEQ ID NO:10), NINJ1 (SEQ ID NO:11), AHNAK2 (SEQ ID NO:12), CATSPERB (SEQ ID NO:13), PCNXL4 (SEQ ID NO:14), SYNE2 (SEQ ID NO:15), NLRP12 (SEQ ID NO:16), LOC126086768 (SEQ ID NO:17), WDR90 (SEQ ID NO:18), BRCA2 (SEQ ID NO:19), PRKDC (SEQ ID NO:20), VPS13B (SEQ ID NO:21), PSAP (SEQ ID NO:22), SEC31B (SEQ ID NO:23), KRT28 (SEQ ID NO:24), KRT35 (SEQ ID NO:25), KRT40 (SEQ ID NO:26), MYH4 (SEQ ID NO:27), PIRT (SEQ ID NO:28), PKD1L2 (SEQ ID NO:29), RP1L1 (SEQ ID NO:30), CXorf58 (SEQ ID NO:31), LOC126068772 (SEQ ID NO:32), LOC126069872 (SEQ ID NO:33), THRAP3 (SEQ ID NO:34), CENPC1 (SEQ ID NO:35), DSPP (SEQ ID NO:36), FGF5 (SEQ ID NO:37), CATSPERG (SEQ ID NO:38), MYH1 (SEQ ID NO:39), MYH13 (SEQ ID NO:40), ATRIP (SEQ ID NO:41), and TGM3 (SEQ ID NO:42).

3. An isolated vector comprising the isolated nucleic acid sequence of claim 1.

4. A recombinant host cell comprising at least one isolated nucleic acid sequence of claim 1.

5. A recombinant host cell comprising a nucleotide sequence having at least 80%, at least 85%, at least 90%, at least 95%, at least 96%, at least 97%, at least 98%, or at least 99% identity to at least one of GPR98 (SEQ ID NO:1), MACF1 (SEQ ID NO:2), ADARB2 (SEQ ID NO:3), CEP290 (SEQ ID NO:4), KRT4 (SEQ ID NO:5), LOC126076011 (SEQ ID NO:6), NCKAP5 (SEQ ID NO:7), LAMB4 (SEQ ID NO:8), NPC1L1 (SEQ ID NO:9), ADGRD2 (SEQ ID NO:10), NINJ1 (SEQ ID NO:11), AHNAK2 (SEQ ID NO:12), CATSPERB (SEQ ID NO:13), PCNXL4 (SEQ ID NO:14), SYNE2 (SEQ ID NO:15), NLRP12 (SEQ ID NO:16), LOC126086768 (SEQ ID NO:17), WDR90 (SEQ ID NO:18), BRCA2 (SEQ ID NO:19), PRKDC (SEQ ID NO:20), VPS13B (SEQ ID NO:21), PSAP (SEQ ID NO:22), SEC31B (SEQ ID NO:23), KRT28 (SEQ ID NO:24), KRT35 (SEQ ID NO:25), KRT40 (SEQ ID NO:26), MYH4 (SEQ ID NO:27), PIRT (SEQ ID NO:28), PKD1L2 (SEQ ID NO:29), RP1L1 (SEQ ID NO:30), CXorf58 (SEQ ID NO:31), LOC126068772 (SEQ ID NO:32), LOC126069872 (SEQ ID NO:33), THRAP3 (SEQ ID NO:34), CENPC1 (SEQ ID NO:35), DSPP (SEQ ID NO:36), FGF5 (SEQ ID NO:37), CATSPERG (SEQ ID NO:38), MYH1 (SEQ ID NO:39), MYH13 (SEQ ID NO:40), ATRIP (SEQ ID NO:41), and TGM3 (SEQ ID NO:42).

6. The recombinant host cell of claim 5, wherein the host cell comprises a nucleotide sequence having at least one of GPR98 (SEQ ID NO:1), MACF1 (SEQ ID NO:2), ADARB2 (SEQ ID NO:3), CEP290 (SEQ ID NO:4), KRT4 (SEQ ID NO:5), LOC126076011 (SEQ ID NO:6), NCKAP5 (SEQ ID NO:7), LAMB4 (SEQ ID NO:8), NPC1L1 (SEQ ID NO:9), ADGRD2 (SEQ ID NO:10), NINJ1 (SEQ ID NO:11), AHNAK2 (SEQ ID NO:12), CATSPERB (SEQ ID NO:13), PCNXL4 (SEQ ID NO:14), SYNE2 (SEQ ID NO:15), NLRP12 (SEQ ID NO:16), LOC126086768 (SEQ ID NO:17), WDR90 (SEQ ID NO:18), BRCA2 (SEQ ID NO:19), PRKDC (SEQ ID NO:20), VPS13B (SEQ ID NO:21), PSAP (SEQ ID NO:22), SEC31B (SEQ ID NO:23), KRT28 (SEQ ID NO:24), KRT35 (SEQ ID NO:25), KRT40 (SEQ ID NO:26), MYH4 (SEQ ID NO:27), PIRT (SEQ ID NO:28), PKD1L2 (SEQ ID NO:29), RP1L1 (SEQ ID NO:30), CXorf58 (SEQ ID NO:31), LOC126068772 (SEQ ID NO:32), LOC126069872 (SEQ ID NO:33), THRAP3 (SEQ ID NO:34), CENPC1 (SEQ ID NO:35), DSPP (SEQ ID NO:36), FGF5 (SEQ ID NO:37), CATSPERG (SEQ ID NO:38), MYH1 (SEQ ID NO:39), MYH13 (SEQ ID NO:40), ATRIP (SEQ ID NO:41), and TGM3 (SEQ ID NO:42).

7. The recombinant host cell of claim 4, wherein the recombinant host cell comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 isolated nucleic acid sequences.

8. A recombinant host cell comprising a deletion of at least one nucleotide sequence upstream of a transcription start site of LOC126071805, WASH complex subunit 4 (WASHC4), LOC126075532, LOC126075533, alpha-fetoprotein (AFP), LOC126079103, LOC126079327, LOC126079327, LOC126080333, LOC126080367, LOC126080369, LOC126080369, LOC126080733, distal-less homeobox 6 (DLX6), extracellular matrix protein 2 (ECM2), LOC126085059, LOC126085481, LOC126086151, LOC126086152, LOC126086293, LOC126058227, LOC126058390, LOC126058396, forkhead box H1 (FOXH1), LOC126060018, LOC126060570, coiled-coil domain containing 15 (CCDC15), INO80 complex subunit B (INO80B), LOC126062579, LOC126063153, LOC126063990, LOC126063991, LOC126066513, LOC126066877, acyl-CoA wax alcohol acyltransferase 1 (AWAT1), transmembrane protein 187 (TMEM187), and LOC126069912, wherein the deleted nucleic acid sequence upstream of:
(a) LOC126071805 comprises SEQ ID NO:43,
(b) WASHC4 comprises SEQ ID NO:44,
(c) LOC126075532 comprises SEQ ID NO:45,
(d) LOC126075533 comprises SEQ ID NO:46,
(e) AFP comprises SEQ ID NO:47,
(f) LOC126079103 comprises SEQ ID NO:48,
(g) LOC126079327 comprises SEQ ID NO:49,
(h) LOC126079327 comprises SEQ ID NO:50,
(i) LOC126080333 comprises SEQ ID NO:51,
(j) LOC126080367 comprises SEQ ID NO:52,
(k) LOC126080369 comprises SEQ ID NO:53,
(l) LOC126080369 comprises SEQ ID NO:54,
(m) LOC126080733 comprises SEQ ID NO:55,
(n) DLX6 comprises SEQ ID NO:56,
(o) ECM2 comprises SEQ ID NO:57,
(p) LOC126085059 comprises SEQ ID NO:58,
(q) LOC126085481 comprises SEQ ID NO:59,
(r) LOC126086151 comprises SEQ ID NO:60,
(s) LOC126086152 comprises SEQ ID NO:61,
(t) LOC126086293 comprises SEQ ID NO:62,
(u) LOC126058227 comprises SEQ ID NO:63,
(v) LOC126058390 comprises SEQ ID NO:64,
(w) LOC126058396 comprises SEQ ID NO:65,
(x) FOXH1 comprises SEQ ID NO:66,
(y) LOC126060018 comprises SEQ ID NO:67,
(z) LOC126060570 comprises SEQ ID NO:68,
(aa) CCDC15 comprises SEQ ID NO:69,
(bb) INO80B comprises SEQ ID NO:70,
(cc) LOC126062579 comprises SEQ ID NO:71,
(dd) LOC126063153 comprises SEQ ID NO:72,
(ee) LOC126063990 comprises SEQ ID NO:73,
(ff) LOC126063991 comprises SEQ ID NO:74,
(gg) LOC126066513 comprises SEQ ID NO:75,
(hh) LOC126066877 comprises SEQ ID NO:76,
(ii) AWAT1 comprises SEQ ID NO:77,
(jj) TMEM187 comprises SEQ ID NO:78, and
(kk) LOC126069912 comprises SEQ ID NO:79.

9. The recombinant host cell of claim 7, further comprising a deletion of at least one nucleotide sequence upstream of a transcription start site of LOC126071805, WASHC4, LOC126075532, LOC126075533, AFP, LOC126079103, LOC126079327, LOC126079327, LOC126080333, LOC126080367, LOC126080369, LOC126080369, LOC126080733, DLX6, ECM2, LOC126085059, LOC126085481, LOC126086151, LOC126086152, LOC126086293, LOC126058227, LOC126058390, LOC126058396, FOXH1, LOC126060018, LOC126060570, CCDC15, INO80B, LOC126062579, LOC126063153, LOC126063990, LOC126063991, LOC126066513, LOC126066877, AWAT1, TMEM187, and LOC126069912, wherein the deleted nucleic acid sequence upstream of:
(a) LOC126071805 comprises SEQ ID NO:43,
(b) WASHC4 comprises SEQ ID NO:44,
(c) LOC126075532 comprises SEQ ID NO:45,
(d) LOC126075533 comprises SEQ ID NO:46,
(e) AFP comprises SEQ ID NO:47,
(f) LOC126079103 comprises SEQ ID NO:48,
(g) LOC126079327 comprises SEQ ID NO:49,
(h) LOC126079327 comprises SEQ ID NO:50,
(i) LOC126080333 comprises SEQ ID NO:51,
(j) LOC126080367 comprises SEQ ID NO:52,
(k) LOC126080369 comprises SEQ ID NO:53,
(l) LOC126080369 comprises SEQ ID NO:54,
(m) LOC126080733 comprises SEQ ID NO:55,
(n) DLX6 comprises SEQ ID NO:56,
(o) ECM2 comprises SEQ ID NO:57,
(p) LOC126085059 comprises SEQ ID NO:58,
(q) LOC126085481 comprises SEQ ID NO:59,
(r) LOC126086151 comprises SEQ ID NO:60,
(s) LOC126086152 comprises SEQ ID NO:61,
(t) LOC126086293 comprises SEQ ID NO:62,
(u) LOC126058227 comprises SEQ ID NO:63,
(v) LOC126058390 comprises SEQ ID NO:64,
(w) LOC126058396 comprises SEQ ID NO:65,
(x) FOXH1 comprises SEQ ID NO:66,
(y) LOC126060018 comprises SEQ ID NO:67,
(z) LOC126060570 comprises SEQ ID NO:68,
(aa) CCDC15 comprises SEQ ID NO:69,
(bb) INO80B comprises SEQ ID NO:70,
(cc) LOC126062579 comprises SEQ ID NO:71,
(dd) LOC126063153 comprises SEQ ID NO:72,
(ee) LOC126063990 comprises SEQ ID NO:73,
(ff) LOC126063991 comprises SEQ ID NO:74,
(gg) LOC126066513 comprises SEQ ID NO:75,
(hh) LOC126066877 comprises SEQ ID NO:76,
(ii) AWAT1 comprises SEQ ID NO:77,
(jj) TMEM187 comprises SEQ ID NO:78, and
(kk) LOC126069912 comprises SEQ ID NO:79.

10. The recombinant host cell of claim 4, wherein the recombinant host cell is:
(a) a stem cell;
(b) a reprogrammed cell;
(c) a fibroblast cell or a mesenchymal cell; or
(d) selected from the group consisting of a nerve cell, a cartilage cell, a bone cell, a muscle cell, a bone cell, a fat cell, and an epidermal cell.

11. The recombinant host cell of claim 10, wherein the stem cell is selected from an induced stem cell, embryonic stem (ES) cell, or a mesenchymal stem cell (MSC).

12-14. (canceled)

15. The recombinant host cell of claim 4, wherein the recombinant host cell fails to express an endogenous homologue of at least one of GPR98, MACF1, ADARB2, CEP290, KRT4, LOC126076011, NCKAP5, LAMB4, NPC1L1, ADGRD2, NINJ1, AHNAK2, CATSPERB, PCNXL4, SYNE2, NLRP12, LOC126086768, WDR90, BRCA2, PRKDC, VPS13B, PSAP, SEC31B, KRT28, KRT35, KRT40, MYH4, PIRT, PKD1L2, RP1L1, CXorf58, LOC126068772, LOC126069872, THRAP3, CENPC1, DSPP, FGF5, CATSPERG, MYH1, MYH13, ATRIP, and TGM3.

16. The recombinant host cell of claim 15, wherein the recombinant host cell fails to express the endogenous homologue of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 of GPR98, MACF1, ADARB2, CEP290, KRT4, LOC126076011, NCKAP5, LAMB4, NPC1L1, ADGRD2, NINJ1, AHNAK2, CATSPERB, PCNXL4, SYNE2, NLRP12, LOC126086768, WDR90, BRCA2, PRKDC, VPS13B, PSAP, SEC31B, KRT28, KRT35, KRT40, MYH4, PIRT, PKD1L2, RP1L1, CXorf58, LOC126068772, LOC126069872, THRAP3, CENPC1, DSPP, FGF5, CATSPERG, MYH1, MYH13, ATRIP, and TGM3.

17. The recombinant host cell of claim 4, wherein the recombinant host cell is an elephant cell.

18. The recombinant host cell of claim 17, wherein the elephant cell is selected from an Asian elephant cell (Elephas maximus), an African elephant cell (Loxodonta africana), an African forest elephant cell (Loxodonta cyclotis), and a Bornean elephant cell (Elephas maximus borneensis).

19. A transgenic animal comprising the recombinant host cell of claim 4.

20. The transgenic animal of claim 19, wherein the transgenic animal is an Asian elephant (Elephas maximus), an African elephant (Loxodonta africana), an African forest elephant (Loxodonta cyclotis), and a Bornean elephant (Elephas maximus borneensis).

21. A transgenic animal comprising at least one woolly mammoth (Mammuthus primigenius) gene variant.

22. The transgenic animal of claim 21, wherein the at least one woolly mammoth (Mammuthus primigenius) gene variant comprises a nucleotide sequence encoding an amino acid sequence having at least 80%, at least 85%, at least 90%, at least 95%, at least 96%, at least 97%, at least 98%, or at least 99% identity to at least one of G protein-coupled receptor 98 (GPR98) (SEQ ID NO:1), microtubule actin crosslinking factor 1 (MACF1) (SEQ ID NO:2), adenosine deaminase RNA specific B2 (ADARB2) (SEQ ID NO:3), centrosomal protein 290 (CEP290) (SEQ ID NO:4), keratin 4 (KRT4) (SEQ ID NO:5), LOC126076011 (SEQ ID NO:6), NCK associated protein 5 (NCKAP5) (SEQ ID NO:7), laminin subunit beta 4 (LAMB4) (SEQ ID NO:8), Niemann-Pick C1-like 1 (NPC1L1) (SEQ ID NO:9), adhesion G protein-coupled receptor D2 (ADGRD2) (SEQ ID NO:10), ninjurin 1 (NINJ1) (SEQ ID NO:11), AHNAK nucleoprotein 2 (AHNAK2) (SEQ ID NO:12), cation channel sperm associated auxiliary subunit beta (CATSPERB) (SEQ ID NO:13), pecanex-like 4 (PCNXL4) (SEQ ID NO:14), spectrin repeat containing nuclear envelope protein 2 (SYNE2) (SEQ ID NO:15), NLR family pyrin domain containing 12 (NLRP12) (SEQ ID NO:16), LOC126086768 (SEQ ID NO:17), WD repeat domain 90 (WDR90) (SEQ ID NO:18), breast cancer 2 (BRCA2) (SEQ ID NO:19), protein kinase, DNA-activated catalytic subunit (PRKDC) (SEQ ID NO:20), vacuolar protein sorting 13 homolog B (VPS13B) (SEQ ID NO:21), prosaposin (PSAP) (SEQ ID NO:22), SEC31 homolog B (SEC31B) (SEQ ID NO:23), keratin 28 (KRT28) (SEQ ID NO:24), keratin 35 (KRT35) (SEQ ID NO:25), keratin 40 (KRT40) (SEQ ID NO:26), myosin heavy chain 4 (MYH4) (SEQ ID NO:27), phosphoinositide interacting regulator of TRP (PIRT) (SEQ ID NO:28), polycystin 1 like 2 (PKD1L2) (SEQ ID NO:29), retinitis pigmentosa 1-like protein (RP1L1) (SEQ ID NO:30), chromosome X open reading frame 58 (CXorf58) (SEQ ID NO:31), LOC126068772 (SEQ ID NO:32), LOC126069872 (SEQ ID NO:33), thyroid hormone receptor associated protein 3 (THRAP3) (SEQ ID NO:34), centromere protein C 1 (CENPC1) (SEQ ID NO:35), dentinogenesis and dentin sialophosphoprotein (DSPP) (SEQ ID NO:36), fibroblast growth factor 5 (FGF5) (SEQ ID NO:37), cation channel sperm associated auxiliary subunit gamma (CATSPERG) (SEQ ID NO:38), myosin heavy chain 1 (MYH1) (SEQ ID NO:39), myosin heavy chain 13 (MYH13) (SEQ ID NO:40), ATR interacting protein (ATRIP) (SEQ ID NO:41), and transglutaminase 3 (TGM3) (SEQ ID NO:42).

23. The transgenic animal of claim 22, wherein the at least one woolly mammoth (Mammuthus primigenius) gene variant comprises at least one of GPR98 (SEQ ID NO:1), MACF1 (SEQ ID NO:2), ADARB2 (SEQ ID NO:3), CEP290 (SEQ ID NO:4), KRT4 (SEQ ID NO:5), LOC126076011 (SEQ ID NO:6), NCKAP5 (SEQ ID NO:7), LAMB4 (SEQ ID NO:8), NPC1L1 (SEQ ID NO:9), ADGRD2 (SEQ ID NO:10), NINJ1 (SEQ ID NO: 11), AHNAK2 (SEQ ID NO:12), CATSPERB (SEQ ID NO:13), PCNXL4 (SEQ ID NO:14), SYNE2 (SEQ ID NO:15), NLRP12 (SEQ ID NO:16), LOC126086768 (SEQ ID NO:17), WDR90 (SEQ ID NO:18), BRCA2 (SEQ ID NO:19), PRKDC (SEQ ID NO:20), VPS13B (SEQ ID NO:21), PSAP (SEQ ID NO:22), SEC31B (SEQ ID NO:23), KRT28 (SEQ ID NO:24), KRT35 (SEQ ID NO:25), KRT40 (SEQ ID NO:26), MYH4 (SEQ ID NO:27), PIRT (SEQ ID NO:28), PKD1L2 (SEQ ID NO:29), RP1L1 (SEQ ID NO:30), CXorf58 (SEQ ID NO:31), LOC126068772 (SEQ ID NO:32), LOC126069872 (SEQ ID NO:33), THRAP3 (SEQ ID NO:34), CENPC1 (SEQ ID NO:35), DSPP (SEQ ID NO:36), FGF5 (SEQ ID NO:37), CATSPERG (SEQ ID NO:38), MYH1 (SEQ ID NO:39), MYH13 (SEQ ID NO:40), ATRIP (SEQ ID NO:41), and TGM3 (SEQ ID NO:42).

24. The transgenic animal of claim 21, wherein the transgenic animal comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 woolly mammoth (Mammuthus primigenius) gene variants.

25. The transgenic animal of claim 21, wherein the transgenic animal further comprises a deletion of at least one nucleotide sequence upstream of a transcription start site of LOC126071805, WASH complex subunit 4 (WASHC4), LOC126075532, LOC126075533, alpha-fetoprotein (AFP), LOC126079103, LOC126079327, LOC126079327, LOC126080333, LOC126080367, LOC126080369, LOC126080369, LOC126080733, distal-less homeobox 6 (DLX6), extracellular matrix protein 2 (ECM2), LOC126085059, LOC126085481, LOC126086151, LOC126086152, LOC126086293, LOC126058227, LOC126058390, LOC126058396, forkhead box H1 (FOXH1), LOC126060018, LOC126060570, coiled-coil domain containing 15 (CCDC15), INO80 complex subunit B (INO80B), LOC126062579, LOC126063153, LOC126063990, LOC126063991, LOC126066513, LOC126066877, acyl-CoA wax alcohol acyltransferase 1 (AWAT1), transmembrane protein 187 (TMEM187), and LOC126069912, wherein the deleted nucleic acid sequence upstream of:
(a) LOC126071805 comprises SEQ ID NO:43,
(b) WASHC4 comprises SEQ ID NO:44,
(c) LOC126075532 comprises SEQ ID NO:45,
(d) LOC126075533 comprises SEQ ID NO:46,
(e) AFP comprises SEQ ID NO:47,
(f) LOC126079103 comprises SEQ ID NO:48,
(g) LOC126079327 comprises SEQ ID NO:49,
(h) LOC126079327 comprises SEQ ID NO:50,
(i) LOC126080333 comprises SEQ ID NO:51,
(j) LOC126080367 comprises SEQ ID NO:52,
(k) LOC126080369 comprises SEQ ID NO:53,
(l) LOC126080369 comprises SEQ ID NO:54,
(m) LOC126080733 comprises SEQ ID NO:55,
(n) DLX6 comprises SEQ ID NO:56,
(o) ECM2 comprises SEQ ID NO:57,
(p) LOC126085059 comprises SEQ ID NO:58,
(q) LOC126085481 comprises SEQ ID NO:59,
(r) LOC126086151 comprises SEQ ID NO:60,
(s) LOC126086152 comprises SEQ ID NO:61,
(t) LOC126086293 comprises SEQ ID NO:62,
(u) LOC126058227 comprises SEQ ID NO:63,
(v) LOC126058390 comprises SEQ ID NO:64,
(w) LOC126058396 comprises SEQ ID NO:65,
(x) FOXH1 comprises SEQ ID NO:66,
(y) LOC126060018 comprises SEQ ID NO:67,
(z) LOC126060570 comprises SEQ ID NO:68,
(aa) CCDC15 comprises SEQ ID NO:69,
(bb) INO80B comprises SEQ ID NO:70,
(cc) LOC126062579 comprises SEQ ID NO:71,
(dd) LOC126063153 comprises SEQ ID NO:72,
(ee) LOC126063990 comprises SEQ ID NO:73,
(ff) LOC126063991 comprises SEQ ID NO:74,
(gg) LOC126066513 comprises SEQ ID NO:75,
(hh) LOC126066877 comprises SEQ ID NO:76,
(ii) AWAT1 comprises SEQ ID NO:77,
(jj) TMEM187 comprises SEQ ID NO:78, and
(kk) LOC126069912 comprises SEQ ID NO:79.

26. The transgenic animal of claim 21, wherein the transgenic animal fails to express an endogenous homologue of at least one of GPR98, MACF1, ADARB2, CEP290, KRT4, LOC126076011, NCKAP5, LAMB4, NPC1L1, ADGRD2, NINJ1, AHNAK2, CATSPERB, PCNXL4, SYNE2, NLRP12, LOC126086768, WDR90, BRCA2, PRKDC, VPS13B, PSAP, SEC31B, KRT28, KRT35, KRT40, MYH4, PIRT, PKD1L2, RP1L1, CXorf58, LOC126068772, LOC126069872, THRAP3, CENPC1, DSPP, FGF5, CATSPERG, MYH1, MYH13, ATRIP, and TGM3.

27. The transgenic animal of claim 26, wherein the transgenic animal fails to express the endogenous homologue of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 of GPR98, MACF1, ADARB2, CEP290, KRT4, LOC126076011, NCKAP5, LAMB4, NPC1L1, ADGRD2, NINJ1, AHNAK2, CATSPERB, PCNXL4, SYNE2, NLRP12, LOC126086768, WDR90, BRCA2, PRKDC, VPS13B, PSAP, SEC31B, KRT28, KRT35, KRT40, MYH4, PIRT, PKD1L2, RP1L1, CXorf58, LOC126068772, LOC126069872, THRAP3, CENPC1, DSPP, FGF5, CATSPERG, MYH1, MYH13, ATRIP, and TGM3.

28. The transgenic animal of claim 21, wherein the transgenic animal is an elephant.

29. The transgenic animal of claim 28, wherein the elephant is selected from an Asian elephant (Elephas maximus), an African elephant (Loxodonta africana), an African forest elephant (Loxodonta cyclotis), and a Bornean elephant (Elephas maximus borneensis).
